(19) United States
(12) Patent Application Publication
(10) Pub. No.: US 2021/0261987 A1
(43) Pub. Date: Aug. 26, 2021

(54) PRODUCTION OF ETHANOL WITH ONE OR MORE CO-PRODUCTS IN YEAST

(71) Applicant: Braskem S.A., Camacari (BR)

(72) Inventors: Felipe GALZERANI, Campinas (BR); Adler Gomes MOURA, Campinas (BR); Mateus Schreiner GARCEZ LOPES, Camacari (BR); Luige Armando Lierena CALDERON, Sao Paulo (BR)

(21) Appl. No.: 17/182,061

(22) Filed: Feb. 22, 2021

Related U.S. Application Data
(60) Provisional application No. 63/040,445, filed on Jun. 17, 2020.
(60) Provisional application No. 62/979,905, filed on Feb. 21, 2020.

(51) Int. Cl. C12P 7/06; C12P 7/16; C12P 7/28

(52) U.S. Cl. CPC C12P 7/06; C12R 1/865; C12P 7/28; C12P 7/16

(57) ABSTRACT

The disclosure provides processes for the production of ethanol and one or more co-products from a fermentable carbon source. The ethanol and one or more co-products are produced in an ethanol-producing yeast modified to further produce the one or more co-products. The processes involve contacting a fermentable carbon source with the modified yeast in a fermentation medium, fermenting the yeast in the fermentation medium such that the yeast produces ethanol and the one or more co-products from the fermentable carbon source, and isolating the ethanol and the one or more co-products. The modified yeast is an ethanol-producing yeast that produces ethanol in a greater concentration than the one or more co-products. Additionally, the disclosure provides the modified yeast disclosed herein.

### CROSS-REFERENCE TO RELATED APPLICATIONS

This application claims priority to U.S. Provisional Patent Application No. 63/040,445, filed Jun. 17, 2020, and U.S. Provisional Patent Application No. 62/979,905, filed Feb. 21, 2020, each of which is incorporated herein by reference in their entirety.

### BACKGROUND

Industrial production of ethanol can be carried out by fermentation methods using a variety of microorganisms. Process improvements to achieve higher yields and productivity include the use of different feedstock sources and/or the reduction of byproduct production. Exemplary ethanol fermentation processes are described, for example, in U.S. Patent Application Publication No. 2010/0196978, U.S. Patent Application Publication No. 2018/0030483, and Chinese Patent Application Publication No. 101875912 A. Certain Clostridium species are capable of carrying out a fermentation process to produce ethanol, butanol, and acetone (ABE). Exemplary processes involving Clostridium are described, for example in U.S. Patent Application Publication No. 2015/0093796 and U.S. Pat. No. 9,074,173. Ethanol and another product can also be produced by methods where the ethanol and the other product are not produced via fermentation of a single feedstock by the same microorganism. U.S. Patent Application Publication No. 2019/0106720 describes production of ethanol and xylitol where the xylitol is produced from the xylose present in the fermentation broth, while ethanol is produced from starch. U.S. Pat. No. 5,070,016 describes production of methanol from the carbon dioxide byproduct of anaerobic ethanolic fermentation. Other byproducts of ethanol fermentation include animal feed (see, e.g., U.S. Pat. No. 8,603,786), yeast (see, e.g., European Patent No. 1943346 B1), mycoproteins (see, e.g., U.S. Patent Application Publication No. 2017/0226551), and corn oil (see, e.g., U.S. Patent Application Publication No. 2006/0019360).

Therefore, there exists a need in the art for improved methods of producing ethanol with one or more co-products from a single feedstock by the same microorganism.

### SUMMARY

The present disclosure provides processes for the production of industrially important products using ethanol-producing yeast that have been modified to use a portion of a fermentable carbon source to produce the product while continuing to produce ethanol. The present disclosure also provides the modified yeast.

In some embodiments of each or any of the above or below mentioned embodiments, the process for the production of ethanol and one or more co-products comprises: (a) contacting a fermentable carbon source with an ethanol-producing yeast in a fermentation medium; (b) fermenting the yeast in the fermentation medium, wherein the yeast produces ethanol and one or more co-products from the fermentable carbon source, wherein the produced ethanol is present in a greater concentration in mg/mL than the produced co-products; and (c) isolating the ethanol and the one or more co-products wherein the yeast is a recombinant yeast genetically modified to produce the one or more co-products.

In some embodiments of each or any of the above or below mentioned embodiments, the carbon source is glucose or dextrose.

In some embodiments of each or any of the above or below mentioned embodiments, the carbon source is derived from renewable grain sources obtained by saccharification of a starch-based feedstock, such as corn, wheat, rye, barley, oats, rice, or mixtures thereof.

In some embodiments of each or any of the above or below mentioned embodiments, the carbon source is from a renewable sugar, such as sugar cane, sugar beets, cassava, sweet sorghum, or mixtures thereof.

In some embodiments of each or any of the above or below mentioned embodiments, the ethanol-producing yeast is Saccharomyces cerevisiae.

In some embodiments of each or any of the above or below mentioned embodiments, the Saccharomyces cerevisiae is an industrial strain. Suitable industrial ethanol producer strains include, but are not limited to, the S. cerevisiae PE-2, CAT-1 and Red strains. In some embodiments of each or any of the above or below mentioned embodiments, the Saccharomyces cerevisiae is any common strain used in ethanol industry, a typical laboratory strain, or any strain resulting from the typical method of crossing between strains.

In some embodiments of each or any of the above or below mentioned embodiments, the Saccharomyces cerevisiae is an industrial strain already used in existing industrial ethanol processes, wherein such processes are based on sugar cane, sugar beets, or most preferably, corn as a raw material.

In some embodiments of each or any of the above or below mentioned embodiments, the ethanol-producing yeast is modified to downregulate any of the endogenous enzymes related to the natural ethanol producing metabolic pathway, such as PYK1 and/or PDC1 (pyruvate decarboxylase 1). In some embodiments of each or any of the above or below mentioned embodiments, the ethanol-producing yeast is modified to downregulate or delete other endogenous enzymes that are not directly related to or involved in the natural ethanol producing metabolic pathway such as glycerol pathway enzymes and/or acetate pathway enzymes. In some embodiments of each or any of the above or below mentioned embodiments, the ethanol-producing yeast is modified to downregulate the endogenous pyruvate kinase that catalyzes the conversion of phosphoenolpyruvate (PEP) to pyruvate. In some embodiments of each or any of the above or below mentioned embodiments, pyruvate kinase expression is downregulated by at least 10% compared to the level of wild type pyruvate kinase expression, such as at least 20%, at least 30%, at least 40%, at least 50%, at least 60%, at least 70%, at least 80%, or at least 90%. In some embodiments of each or any of the above or below mentioned embodiments, pyruvate kinase activity is downregulated by at least 10% compared to the level of wild type pyruvate kinase activity, such as at least 20%, at least 30%, at least 40%, at least 50%, at least 60%, at least 70%, at least 80%, or at least 90%. In some embodiments of each or any of the above or below mentioned embodiments, the downregulation of endogenous genes is carried out by a weak promoter (either natural or synthetic), natural or synthetic terminators, natural or synthetic transcription factors, degron peptides, iCRISPR, or any other technique known in the art for downregulation of genes in yeast. In some embodiments of each or any of the above or below mentioned embodiments, the endogenous pyruvate kinase under the control of a weak promoter is expressed at a level that is no more than 90% of the level of wild type pyruvate kinase expression, such as no more than 80%, no more than 70%, no more than 60%, no more than 50%, no more than 40%, no more than 30%, no more than 20%, or no more than 10%. In some embodiments of each or any of the above or below mentioned embodiments, the activity of the endogenous pyruvate kinase under the control of a weak promoter is at a level that is no more than 90% of the level of wild type pyruvate kinase activity, such as no more than 80%, no more than 70%, no more than 60%, no more than 50%, no more than 40%, no more than 30%, no more than 20%, or no more than 10%. In some embodiments of each or any of the above or below mentioned embodiments, the weak promoter is pADH1, pCYC1, pSTE5, pREV1, pURA3, pRPLA1, pGAP1, pNUP57, or pMET25. In some embodiments of each or any of the above or below mentioned embodiments, the ethanol-producing yeast is modified to delete the endogenous pyruvate kinase that catalyzes the conversion of phosphoenolpyruvate (PEP) to pyruvate. In some embodiments of each or any of the above or below mentioned embodiments, the ethanol-producing yeast is modified to express an exogenous pyruvate kinase that catalyzes the conversion of phosphoenolpyruvate (PEP) to pyruvate under the control of a weak promoter. In some embodiments of each or any of the above or below mentioned embodiments, the downregulation of exogenous genes is carried out by a week promoter (either natural or synthetic), natural or synthetic terminators, natural or synthetic transcription factors, degron peptides, or any other technique known in the art for downregulation of genes in yeast. In some embodiments of each or any of the above or below mentioned embodiments, the exogenous pyruvate kinase under the control of a weak promoter is expressed at a level that is no more than 90% of the level of wild type pyruvate kinase expression, such as no more than 80%, no more than 70%, no more than 60%, no more than 50%, no more than 40%, no more than 30%, no more than 20%, or no more than 10%. In some embodiments of each or any of the above or below mentioned embodiments, the activity of the exogenous pyruvate kinase under the control of a weak promoter is at a level that is no more than 90% of the level of wild type pyruvate kinase activity, such as no more than 80%, no more than 70%, no more than 60%, no more than 50%, no more than 40%, no more than 30%, no more than 20%, or no more than 10%. In some embodiments of each or any of the above or below mentioned embodiments, the weak promoter is pADH1, pCYC1, pSTE5, pREV1, pURA3, pRPLA1, pGAP1, pNUP57, or pMET25.

In some embodiments of each or any of the above or below mentioned embodiments, the ethanol-producing yeast is modified to express exogenous phosphoenolpyruvate carboxykinase (PEPCK) kinase to redirect carbon flow from PEP to oxaloacetate.

In some embodiments of each or any of the above or below mentioned embodiments, the co-products are produced at non-toxic concentrations for the ethanol-producing yeast.

In some embodiments of each or any of the above or below mentioned embodiments, the recombinant yeast has most of the ethanol fermentation robustness and performance preserved compared to its mother industrial ethanol-producing yeast, enabling its use on already existing industrial ethanol processes.

In some embodiments of each or any of the above or below mentioned embodiments, the produced ethanol is present in an amount of at least 70 wt. % based on a total weight of produced ethanol and co-products, such as at least 75 wt. %, at least 80 wt. %, at least 85 wt. %, at least 90 wt. %, or at least 95 wt. %.

In some embodiments of each or any of the above or below mentioned embodiments, the fermentation is carried out as a batch process, a fed batch process, or a continuous process.

In some embodiments of each or any of the above or below mentioned embodiments, the fermentation is carried out under anaerobic conditions for about 24 to about 96 hours at a temperature of about 15° C. to about 60° C.

In some embodiments of each or any of the above or below mentioned embodiments, the fermentation is carried out under microaerobic conditions for about 24 to about 96 hours at a temperature of about 15° C. to about 60° C.

In some embodiments of each or any of the above or below mentioned embodiments, the fermentation is carried out under aerobic conditions for about 24 to about 96 hours at a temperature of about 15° C. to about 60° C.

In some embodiments of each or any of the above or below mentioned embodiments, the fermentation is carried out in an industrial ethanol plant, preferable in an already-existing industrial ethanol plant.

In some embodiments of each or any of the above or below mentioned embodiments, the one or more co-products are selected from the group consisting of an alcohol other than ethanol; a ketone; a glycol; an ether; an ester; a diamine; a carboxylic acid; an amino acid; a diene, and an alkene.

In some embodiments of each or any of the above or below mentioned embodiments, the one or more co-products are selected from the group consisting of 1-butanol, 2-butanol, isobutanol, methanol, n-propanol, isopropanol, isoamyl alcohol, acetone, methyl ethyl ketone, methyl propionate, 1,3-propanediol, monoethylene glycol, propylene glycol, citric acid, lactic acid, succinic acid, adipic acid, acetic acid, glutamic acid, propionic acid, furan dicarboxylic acid, 2,4 furandicarboxylic acid, 2,5-furandicarboxylic acid, 3-hydroxypropionic acid, acrylic acid, itaconic acid, glutamic acid, ethyl acetate, isopropyl acetate, propyl acetate, isoprenol, 1,3-butanediol, 1,4-butanediol, 2,3-butanediol, diethanolamine, tryptophan, threonine, methionine, lysine, serine, tyrosine, butadiene, isoprene, ethane, and propene. In some embodiments of each or any of the above or below mentioned embodiments, the co-products have low solubility in water and may aggregate or sediment in the bottom of the fermentation broth tank facilitating their separation and purification from the fermentation broth during downstream processing.

In some embodiments of each or any of the above or below mentioned embodiments, isolating the ethanol and the one or more co-products comprises a process selected from distillation, adsorption, crystallization, absorption, electrodialysis, solvent extraction, ion exchange resin chromatography, or a combination thereof.

In some embodiments of each or any of the above or below mentioned embodiments, the process for the production of ethanol and one or more co-products comprises: (a) contacting a fermentable carbon source with an ethanol-producing yeast in a fermentation medium; (b) fermenting the yeast in the fermentation medium, wherein the yeast produces ethanol and one or more low boiling co-products from the fermentable carbon source, wherein the produced ethanol is present in a greater concentration in mg/mL than the produced co-products; and (c) isolating the ethanol and the one or more low boiling co-products; wherein the yeast is a recombinant yeast genetically modified to produce the one or more co-products.

In some embodiments of each or any of the above or below mentioned embodiments, the low boiling co-products have, at a standard pressure of 100 kPa (1 bar), a boiling point of 100° C. or less, such as 99° C. or less, 98° C. or less, 97° C. or less, 95° C. or less, 90° C. or less, 85° C. or less, 80° C. or less, 75° C. or less, 70° C. or less, 65° C. or less, or 60° C. or less. Exemplary low boiling point products include, but are not limited to, 1-propanol (boiling point: 97° C.), 2-propanol (boiling point: 82° C.), acetone (boiling point: 56° C.), methyl ethyl ketone (boiling point: 80° C.), ethyl acetate (boiling point: 77° C.), isopropyl acetate (boiling point: 88° C.), ethane (boiling point: −90° C.), propene (boiling point: −48° C.), and ethanol (boiling point: 78.3° C.).

In some embodiments of each or any of the above or below mentioned embodiments, the one or more low boiling co-products are selected from acetone, 1-propanol, 2-propanol, or a combination thereof.

In some embodiments of each or any of the above or below mentioned embodiments, isolating the ethanol and the one or more low boiling co-products is conducted by sequential distillation units.

In some embodiments of each or any of the above or below mentioned embodiments, the process for the production of ethanol and one or more co-products comprises: (a) contacting a fermentable carbon source with an ethanol-producing yeast in a fermentation medium; (b) fermenting the yeast in the fermentation medium, wherein the yeast produces ethanol and one or more high boiling co-products from the fermentable carbon source, wherein the produced ethanol is present in a greater concentration in mg/mL than the produced co-products; and (c) isolating the ethanol and the one or more high boiling co-products; wherein the yeast is a recombinant yeast genetically modified to produce the one or more high boiling co-products.

In some embodiments of each or any of the above or below mentioned embodiments, the high boiling co-products have, at a standard pressure of 100 kPa (1 bar), a boiling point of more than 100° C., such as more than 105° C., more than 110° C., more than 120° C., more than 130° C., more than 140° C., more than 150° C., more than 160° C., more than 170° C., more than 180° C., more than 190° C., more than 200° C., more than 210° C., more than 220° C., more than 230° C., more than 240° C., or more than 250° C. Exemplary high boiling point products include, but are not limited to, monoethylene glycol (boiling point: 197° C.), n-butanol (boiling point: 118° C.), 3-hydroxypropionic acid (boiling point: 280° C.), adipic acid (boiling point: 338° C.), diethanolamine (boiling point: 268° C.), and 1,3-propanediol (boiling point: 214° C.).

In some embodiments of each or any of the above or below mentioned embodiments, the one or more high boiling co-products are selected from 1-butanol, isobutanol, isoamyl alcohol, or a combination thereof.

In some embodiments of each or any of the above or below mentioned embodiments, isolating the ethanol and the one or more high boiling co-products is conducted by distillation and followed by a process selected from crystallization, solvent extraction, chromatographic separation, salt splitting, sedimentation, acidification, ion exchange, evaporation, or combinations thereof.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 depicts exemplary metabolic pathways for the production of 1-propanol by fermentation.

FIG. 2 depicts exemplary metabolic pathways for the production of acetone, 2-propanol, propene, and 1-butanol by fermentation.

FIG. 3 depicts an exemplary metabolic pathway for the co-production of 1-propanol and acetone or 1-propanol and 2-propanol.

FIG. 4 depicts an exemplary metabolic pathway for the production of butanone and/or 2-butanol.

FIG. 5 depicts an exemplary metabolic pathway for the co-production of 1-propanol and butanone.

FIG. 6 is a graph showing inhibition of sugar consumption at various alcohol concentrations (g/L). Dotted lines: linear regression. Squares: 2-butanol. Triangles: n-propanol. Circles: 2-propanol. Diamonds: ethanol.

FIG. 7 is a graph showing glucose and alcohol concentrations at different time points during fermentation. Continuous lines: Condition 1 (added ethanol). Dotted lines: Condition 2 (added n-propanol and 2-propanol). Filled circle: glucose consumption under Condition 1. Filled square: alcohol production/added under Condition 1. Empty circle: glucose consumption under Condition 2. Empty square: alcohol production/added under Condition 2.

### DETAILED DESCRIPTION

The present disclosure provides modified yeast (e.g., recombinant yeast) and processes using the modified yeast to produce industrially important products. The modified yeast are ethanol-producing yeast modified to use a portion of a fermentable carbon source to produce the product(s) while continuing to produce ethanol. An advantage of the disclosure is the ability to divert only a minor part of the carbon source from ethanol production to the production of products of industrial relevance, thereby facilitating production of target products that are toxic to yeast cells at high amounts. A related advantage is that the impact of diverting a minor part of the carbon source to the co-product(s) has no or only minimal impact on yeast cell growth and yeast performance to ethanol due to the production of the potentially toxic compounds at low concentrations and below the toxic concentration range that could be fermentation-process impeditive. A further advantage of at least partially retaining yeast ethanol performance while utilizing production conditions similar to those required for industrial production, is the ability to use the modified yeast in an existing ethanol production plant. Yet an additional advantage of the disclosure is the ability to have a modified yeast with robustness to industrial requirements and sufficient ethanol production performance.

The present disclosure provides modified yeast (e.g., recombinant yeast) suitable to be used in already existing industrial ethanol processes to produce products of industrial relevance beyond sugar and ethanol. An advantage of the disclosure is the ability of ethanol producers to be able to diversify their portfolio of products and not to be limited to sugar and ethanol production themselves. A related advantage is the ability of producing varied concentrations of target products and ethanol mixtures, depending on the market price of ethanol and the target products of industrial relevance. A further advantage is the ability to divert part of the carbon source from ethanol production to produce products of industrial relevance of higher market price compared to ethanol in order to enhance profitability. Yet an additional advantage of the disclosure is the ability to provide suitable modified yeast to be used in existing industrial ethanol production plants, reducing technical risks, industrialization time and investments regarding a greenfield plant construction and scaling-up processes.

The present disclosure provides modified yeast (e.g., recombinant yeast) capable of diverting a minor part of the carbon source from ethanol production to the production of products of industrial relevance. An advantage of the disclosure is that the modified yeast is minimally modified to be capable of producing products at low amounts compared to ethanol without compromising the requirements of industrial robustness and ethanol performance of the industrial ethanol yeast strain. A related advantage is the ability to leverage modified yeasts in a shorter period of time with reduced research and development program investment because extensive metabolic engineering work is not necessary and fully optimized metabolic pathway enzymes are not required to produce products at such lower concentrations. In contrast, more time-consuming research and development work and increased cost overall would be required to leverage a modified yeast capable of diverting a major part or all carbon source to a desired product that is not ethanol.

As used herein, the term “derived from” may encompass the terms originated from, obtained from, obtainable from, isolated from, and created from, and generally indicates that one specified material finds its origin in another specified material or has features that can be described with reference to the another specified material.

As used herein, “exogenous polynucleotide” refers to any deoxyribonucleic acid that originates outside of the microorganism.

As used herein, the term “an expression vector” may refer to a DNA construct containing a polynucleotide or nucleic acid sequence encoding a polypeptide or protein, such as a DNA coding sequence (e.g. gene sequence) that is operably linked to one or more suitable control sequence(s) capable of affecting expression of the coding sequence in a host. Such control sequences include a promoter to affect transcription, an optional operator sequence to control such transcription, a sequence encoding suitable mRNA ribosome binding sites, and sequences which control termination of transcription and translation. The vector may be a plasmid, cosmid, phage particle, bacterial artificial chromosome, or simply a potential genomic insert. Once transformed into a suitable host, the vector may replicate and function independently of the host genome (e.g., independent vector or plasmid), or may, in some instances, integrate into the genome itself (e.g., integrated vector). The plasmid is the most commonly used form of expression vector. However, the disclosure is intended to include such other forms of expression vectors that serve equivalent functions and which are, or become, known in the art.

As used herein, the term “expression” may refer to the process by which a polypeptide is produced based on a nucleic acid sequence encoding the polypeptides (e.g., a gene). The process includes both transcription and translation.

As used herein, the term “gene” may refer to a DNA segment that is involved in producing a polypeptide or protein (e.g., fusion protein) and includes regions preceding and following the coding regions as well as intervening sequences (introns) between individual coding segments (exons).

As used herein, the term “heterologous,” with reference to a nucleic acid, polynucleotide, protein or peptide, may refer to a nucleic acid, polynucleotide, protein or peptide that does not naturally occur in a specified cell, e.g., a host cell. It is intended that the term encompass proteins that are encoded by naturally occurring genes, mutated genes, and/or synthetic genes. In contrast, the term homologous, with reference to a nucleic acid, polynucleotide, protein or peptide, refers to a nucleic acid, polynucleotide, protein or peptide that occurs naturally in the cell.

As used herein, the term a “host cell” may refer to a cell or cell line, including a cell such as a microorganism which a recombinant expression vector may be transfected for expression of a polypeptide or protein (e.g., fusion protein). Host cells include progeny of a single host cell, and the progeny may not necessarily be completely identical (in morphology or in total genomic DNA complement) to the original parent cell due to natural, accidental, or deliberate mutation. A host cell may include cells transfected or transformed in vivo with an expression vector.

As used herein, the term “introduced,” in the context of inserting a nucleic acid sequence or a polynucleotide sequence into a cell, may include transfection, transformation, or transduction and refers to the incorporation of a nucleic acid sequence or polynucleotide sequence into a eukaryotic or prokaryotic cell wherein the nucleic acid sequence or polynucleotide sequence may be incorporated into the genome of the cell (e.g., chromosome, plasmid, plastid, or mitochondrial DNA), converted into an autonomous replicon, or transiently expressed.

As used herein, the term “non-naturally occurring” or “modified” when used in reference to a microbial organism or microorganism of the invention is intended to mean that the microbial organism has at least one genetic alteration not normally found in a naturally occurring strain of the referenced species, including wild-type strains of the referenced species. Genetic alterations include, for example, modifications introducing expressible nucleic acids encoding metabolic polypeptides, other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Non-naturally occurring microbial organisms of the disclosure can contain stable genetic alterations, which refers to microorganisms that can be cultured for greater than five generations without loss of the alteration. Generally, stable genetic alterations include modifications that persist greater than 10 generations, particularly stable modifications will persist more than about 25 generations, and more particularly, stable genetic modifications will be greater than 50 generations, including indefinitely. Those skilled in the art will understand that the genetic alterations, including metabolic modifications exemplified herein, are described with reference to a suitable host organism and their corresponding metabolic reactions or a suitable source organism for desired genetic material such as genes for a desired metabolic pathway. However, given the complete genome sequencing of a wide variety of organisms and the high level of skill in the area of genomics, those skilled in the art will readily be able to apply the teachings and guidance provided herein to essentially all other organisms. Such genetic alterations include, for example, genetic alterations of species homologs, in general, and in particular, orthologs, paralogs or nonorthologous gene displacements.

As used herein, the term “operably linked” may refer to a juxtaposition or arrangement of specified elements that allows them to perform in concert to bring about an effect. For example, a promoter may be operably linked to a coding sequence if it controls the transcription of the coding sequence.

As used herein, “1-propanol” is intended to mean n-propanol with a general formula CH₃CH₂CH₂OH (CAS number-71-23-8).

As used herein, “2-propanol” is intended to mean isopropyl alcohol with a general formula CH₃CH₃CHOH (CAS number-67-63-0).

As used herein, the term “a promoter” may refer to a regulatory sequence that is involved in binding RNA polymerase to initiate transcription of a gene. A promoter may be an inducible promoter or a constitutive promoter. An inducible promoter is a promoter that is active under environmental or developmental regulatory conditions.

As used herein, the term “a polynucleotide” or “nucleic acid sequence” may refer to a polymeric form of nucleotides of any length and any three-dimensional structure and single- or multi-stranded (e.g., single-stranded, double-stranded, triple-helical, etc.), which contain deoxyribonucleotides, ribonucleotides, and/or analogs or modified forms of deoxyribonucleotides or ribonucleotides, including modified nucleotides or bases or their analogs. Such polynucleotides or nucleic acid sequences may encode amino acids (e.g., polypeptides or proteins such as fusion proteins). Because the genetic code is degenerate, more than one codon may be used to encode a particular amino acid, and the present disclosure encompasses polynucleotides which encode a particular amino acid sequence. Any type of modified nucleotide or nucleotide analog may be used, so long as the polynucleotide retains the desired functionality under conditions of use, including modifications that increase nuclease resistance (e.g., deoxy, 2′-O-Me, phosphorothioates, etc.). Labels may also be incorporated for purposes of detection or capture, for example, radioactive or nonradioactive labels or anchors, e.g., biotin. The term polynucleotide also includes peptide nucleic acids (PNA). Polynucleotides may be naturally occurring or non-naturally occurring. The terms polynucleotide, nucleic acid, and oligonucleotide are used herein interchangeably. Polynucleotides may contain RNA, DNA, or both, and/or modified forms and/or analogs thereof. A sequence of nucleotides may be interrupted by non-nucleotide components. One or more phosphodiester linkages may be replaced by alternative linking groups. These alternative linking groups include, but are not limited to, embodiments wherein phosphate is replaced by P(O)S (thioate), P(S)S (dithioate), (O)NR₂ (amidate), P(O)R, P(O)OR′, COCH₂ (formacetal), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. Polynucleotides may be linear or circular or comprise a combination of linear and circular portions.

As used herein, the term a “protein” or “polypeptide” may refer to a composition comprised of amino acids and recognized as a protein by those of skill in the art. The conventional one-letter or three-letter code for amino acid residues is used herein. The terms protein and polypeptide are used interchangeably herein to refer to polymers of amino acids of any length, including those comprising linked (e.g., fused) peptides/polypeptides (e.g., fusion proteins). The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, related proteins, polypeptides or peptides may encompass variant proteins, polypeptides or peptides. Variant proteins, polypeptides or peptides differ from a parent protein, polypeptide or peptide and/or from one another by a small number of amino acid residues. In some embodiments, the number of different amino acid residues is any of about 1, 2, 3, 4, 5, 10, 20, 25, 30, 35, 40, 45, or 50. In some embodiments, variants differ by about 1 to about 10 amino acids. Alternatively or additionally, variants may have a specified degree of sequence identity with a reference protein or nucleic acid, e.g., as determined using a sequence alignment tool, such as BLAST, ALIGN, and CLUSTAL (see, infra). For example, variant proteins or nucleic acid may have at least about 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% amino acid sequence identity with a reference sequence.

As used herein, the term “recovered,” “isolated,” “purified,” and “separated” may refer to a material (e.g., a protein, peptide, nucleic acid, polynucleotide or cell) that is removed from at least one component with which it is naturally associated. For example, these terms may refer to a material which is substantially or essentially free from components which normally accompany it as found in its native state, such as, for example, an intact biological system.

As used herein, the term “recombinant” may refer to nucleic acid sequences or polynucleotides, polypeptides or proteins, and cells based thereon, that have been manipulated by man such that they are not the same as nucleic acids, polypeptides, and cells as found in nature. Recombinant may also refer to genetic material (e.g., nucleic acid sequences or polynucleotides, the polypeptides or proteins they encode, and vectors and cells comprising such nucleic acid sequences or polynucleotides) that has been modified to alter its sequence or expression characteristics, such as by mutating the coding sequence to produce an altered polypeptide, fusing the coding sequence to that of another coding sequence or gene, placing a gene under the control of a different promoter, expressing a gene in a heterologous organism, expressing a gene at decreased or elevated levels, expressing a gene conditionally or constitutively in manners different from its natural expression profile, and the like.

As used herein, the term “transfection” or “transformation” may refer to the insertion of an exogenous nucleic acid or polynucleotide into a host cell. The exogenous nucleic acid or polynucleotide may be maintained as a non-integrated vector, for example, a plasmid, or alternatively, may be integrated into the host cell genome. The term transfecting or transfection is intended to encompass all conventional techniques for introducing nucleic acid or polynucleotide into host cells. Examples of transfection techniques include, but are not limited to, calcium phosphate precipitation, DEAE-dextranmediated transfection, lipofection, electroporation, and microinjection.

As used herein, the term “transformed,” “stably transformed,” and “transgenic” may refer to a cell that has a non-native (e.g., heterologous) nucleic acid sequence or polynucleotide sequence integrated into its genome or as an episomal plasmid that is maintained through multiple generations.

As used herein, the term “vector” may refer to a polynucleotide sequence designed to introduce nucleic acids into one or more cell types. Vectors include cloning vectors, expression vectors, shuttle vectors, plasmids, phage particles, single and double stranded cassettes and the like.

As used herein, the term “wild-type,” “native,” or “naturally-occurring” proteins may refer to those proteins found in nature. The terms wild-type sequence refers to an amino acid or nucleic acid sequence that is found in nature or naturally occurring. In some embodiments, a wild-type sequence is the starting point of a protein engineering project, for example, production of variant proteins.

As used herein, the term “non-toxic concentrations” may refer to concentrations of a co-product that have no effect or only a minimal effect on the level of ethanol produced by a yeast modified to produce the co-product compared to the level of ethanol produced by an otherwise similar unmodified yeast. For example, when non-toxic concentrations are present, the level of ethanol produced by the modified yeast may be reduced by no more than 30%, 20%, or, most preferably, no more than 10% compared to the level of ethanol produced by an unmodified yeast.

Unless defined otherwise herein, all technical and scientific terms used herein have the same meaning as commonly understood by one of ordinary skill in the art to which this disclosure belongs. Singleton, et al., Dictionary of Microbiology and Molecular Biology, second ed., John Wiley and Sons, New York (1994), and Hale & Markham, The Harper Collins Dictionary of Biology, Harper Perennial, NY (1991) provide one of skill with a general dictionary of many of the terms used in this disclosure. Further, it will be understood that any of the substrates disclosed in any of the pathways herein may alternatively include the anion or the cation of the substrate.

Numeric ranges provided herein are inclusive of the numbers defining the range.

While the present disclosure is capable of being embodied in various forms, the description below of several embodiments is made with the understanding that the present disclosure is to be considered as an exemplification of the disclosure, and is not intended to limit the disclosure to the specific embodiments illustrated. Headings are provided for convenience only and are not to be construed to limit the disclosure in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present disclosure.

### Modification of Yeast

A yeast may be modified (e.g., genetically engineered) by any method known in the art to comprise and/or express one or more polynucleotides coding for enzymes in a pathway that catalyze a conversion of a fermentable carbon source to one or more products.

In some embodiments, a yeast may be modified (e.g., genetically engineered) by any method known in the art to comprise and/or express one or more polynucleotides coding for enzymes in a pathway that catalyze a conversion of a fermentable carbon source to intermediates in a pathway for the production of a co-product such as 1-propanol, acetone, 2-propanol, propene, 1-butanol, 2-butanol, methyl ethyl ketone, and/or methyl propionate. Such enzymes may include, but are not limited to, any of those enzymes as described herein. For example, the yeast may be modified to comprise one or more polynucleotides coding for enzymes that catalyze a conversion of succinyl-CoA to 1-propanol.

In some embodiments, the yeast may comprise one or more exogenous polynucleotides encoding one or more enzymes in pathways for the production of the product(s), such as 1-propanol, acetone, 2-propanol, propene, 1-butanol, 2-butanol, methyl ethyl ketone, and/or methyl propionate, from a fermentable carbon source under anaerobic conditions.

### Pathways for Production of 1-Propanol

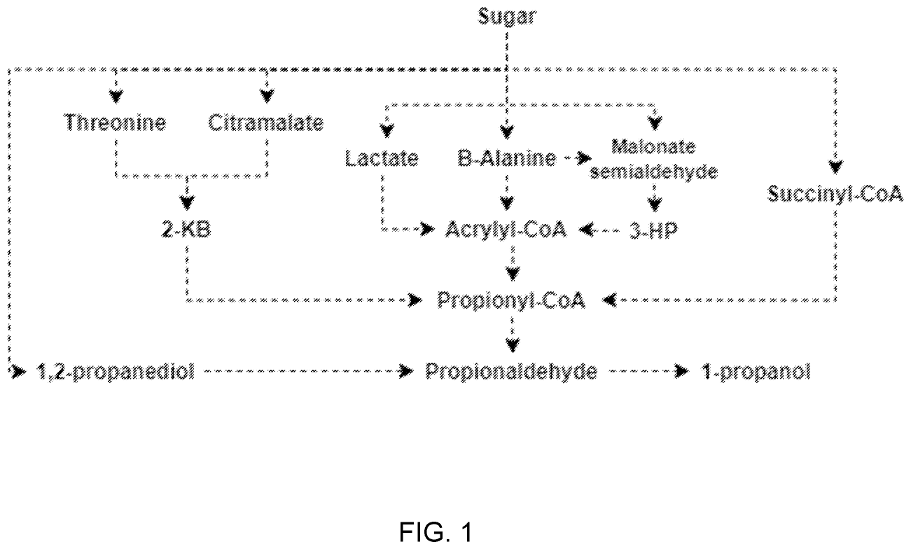

Metabolic pathways for the production of 1-propanol include pathways that produce 1-propanol from intermediates including, but not limited to, malonate semialdehyde, 3-hydroxypropionic acid, 1,2-propanediol, 2-ketobutyrate (2-kB), succinyl-CoA, and acrylyl-CoA. As shown in FIG. 1, the 2-kB, succinyl-CoA, and acrylyl-CoA intermediates converge into propionyl-CoA. Both propionyl-CoA and 1,2-propanediol are converted to propionaldehyde and to 1-propanol by a bi-functional aldehyde/alcohol dehydrogenase or by the action of an aldehyde dehydrogenase (acetylating) in combination with an alcohol dehydrogenase.

In one pathway, 1-propanol is produced via the succinyl-CoA route whereby a sugar source is converted to succinyl-CoA via glycolysis and the citric acid cycle (TCA cycle), followed by the isomerization of succinyl-CoA to methylmalonyl-CoA by a methylmalonyl-CoA mutase, and the decarboxylation of methylmalonyl-CoA to propionyl-CoA by a methylmalonyl-CoA decarboxylase. Aldehyde and alcohol dehydrogenases catalyze additional conversions to convert propionyl-CoA to propionaldehyde and propionaldehyde to 1-propanol (see, e.g., U.S. Patent Application Publication No. 2013/0280775). In another pathway, 1-propanol is produced via 1,2-propanediol whereby a sugar source undergoes multiple conversions catalyzed by a methylglyoxal synthase, an aldo-ketoreductase or a glyoxylate reductase and an aldehyde reductase. Hydrolase and dehydrogenases catalyze additional conversions to convert 1,2-propanediol to propanal and propanal to 1-propanol (see, e.g., U.S. Pat. No. 9,957,530).

In another pathway, 1-propanol is produced from a 2-kB intermediate via conversions from threonine and/or citramalate. For example, 2-kB can be converted to propionyl-CoA or directly to propionaldehyde by a 2-oxobutanoate dehydrogenase or a 2-oxobutanoate decarboxylase, respectively (see, e.g., U.S. Patent Application Publication No. 2014/0377820).

In other pathways, 1-propanol is produced from β-alanine, oxaloacetate, lactate, or 3-hydroxypropionate (3-HP) intermediates that are converge to acrylyl-CoA, which is converted to propionyl-CoA by an acrylyl-CoA reductase (see, e.g., U.S. Patent Application Publication No. 2014/0377820). As described above, propionyl-CoA can be converted to 1-propanol by aldehyde and alcohol dehydrogenases.

Pathways for Production of 1-Propanol, Acetone, 2-Propanol, Propene, and/or 1-Butanol

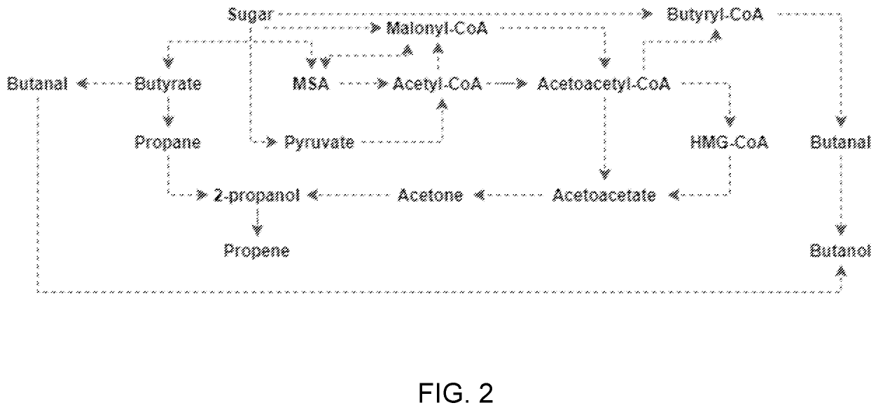

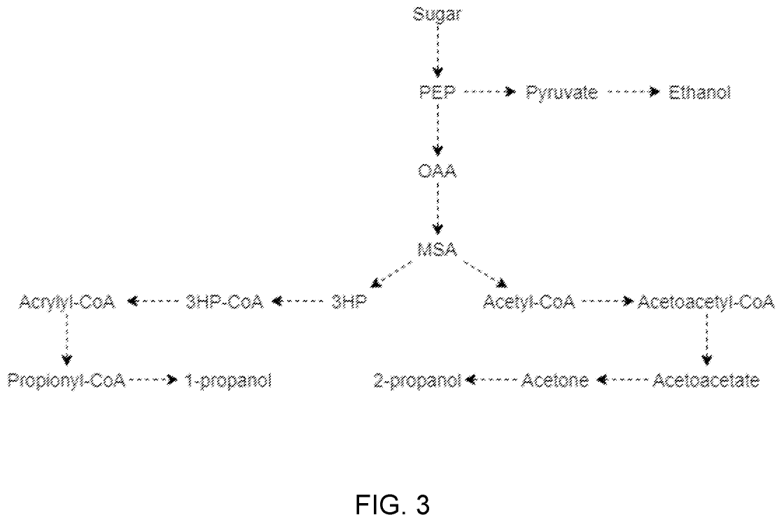

Metabolic pathways for the production of 1-propanol, acetone, 2-propanol, propene, and/or 1-butanol are shown in FIG. 2 and FIG. 3. Acetone can be generated from several pathways, including but not limited to primary and secondary metabolism reactions, as glycolysis, terpenoid biosynthesis, atrazine degradation and cyanoamino acid metabolism. In one pathway, acetyl-CoA can be derived from pyruvate and/or malonate semialdehyde by a pyruvate dehydrogenase and a malonate semialdehyde dehydrogenase, respectively. Acetyl-CoA is converted to acetoacetyl-CoA by a thiolase or an acetyl-CoA acetyltransferase (see, e.g., U.S. Patent Application Publication No. 2018/0179558). Alternatively, acetoacetyl-CoA can be formed through malonyl-CoA by acetoacetyl-CoA synthase. Once acetoacetyl-CoA is formed, its conversion to acetoacetate can be done by an acetoacetyl-CoA transferase or through HMG-CoA by hydroxymethylglutaryl-CoA synthase and hydroxymethylglutaryl-CoA lyase. Acetoacetate conversion to acetone is done by an acetoacetate decarboxylase.

In another pathway, 2-propanol is produced from propane and/or acetone as precursors. As described above, acetone is generated from acetyl-CoA by multiple reactions and is converted to isopropanol by an isopropanol dehydrogenase (see, e.g., U.S. Patent Application Publication No. 2018/0179558). In another pathway, propane is produced from a butyrate intermediate and isopropanol is generated by a propane 2-monooxygenase. Biosynthesis of propane in Escherichia coli from glucose having butyrate as intermediate is described in Kallio et al. (2014) Nat Commun, 5 (4731).

In another pathway, alkenes (e.g., ethene and propene) are produced from alcohol intermediates (e.g., ethanol and propanol, respectively) by a linalool dehydratase-isomerase as described in U.S. Patent Application Publication No. 2019/0323016.

In another pathway, 1-butanol is produced from butanal by a butanol dehydrogenase having butyrate and butyryl-CoA as precursors. Butyryl-ACP is generated via the fatty acid biosynthesis (FASII) pathway, followed by the release of butyrate by thioesterase and its conversion into butanal by carboxylic acid reductase with the aid of a maturase phosphopantetheinyl transferase as described, e.g., in Kallio et al. (2014) Nat Commun, 5 (4731). Butyryl-CoA is produced from crotonyl-CoA by the reaction of a butyryl-CoA dehydrogenase, where the crotonyl-CoA is generated by amino acid metabolism and/or glycolysis via acetyl-CoA as described, e.g., in Ferreira et al. (2019) Biotechnol Biofuels 12:230 and U.S. Pat. No. 9,567,613.

Pathways for Production of Methyl Ethyl Ketone (Butanone) and/or 2-Butanol

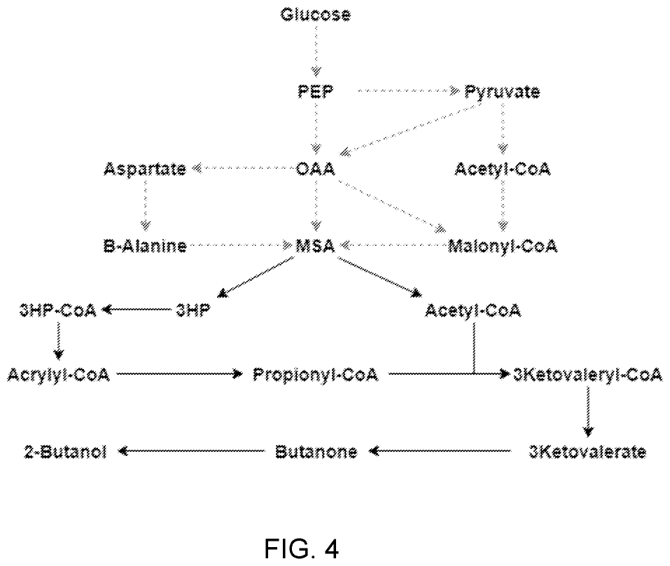

In another pathway, methyl ethyl ketone (also known as butanone) and/or 2-butanol are produced from malonate semialdehyde (MSA) as shown FIG. 4. Metabolic pathways for the production of butanone and/or 2-butanol include pathways that produce butanone and/or 2-butanol from intermediates including, but not limited to, malonate semialdehyde, 3-hydroxypropionic acid (3HP), 3-hydroxypropionyl-coenzyme A (3HP-CoA), acrylyl-CoA, propionyl-CoA, acetyl-CoA, 3-ketovaleryl-CoA, and 3-ketovalerate.

In some aspects, the modified yeast comprises: (a) at least one nucleic acid molecule encoding one or more polypeptides that catalyze the production of acetyl-CoA from malonate semialdehyde; (b) at least one nucleic acid molecule encoding a polypeptide that catalyzes the production of 3-hydroxypropionic acid from malonate semialdehyde; (c) at least one nucleic acid molecule encoding one or more polypeptides that catalyze the production of propionyl-CoA from 3-hydroxypropionic acid; and (d) at least one nucleic acid molecule encoding one or more polypeptides that catalyze the production of 2-butanone from propionyl-CoA and acetyl-CoA.

In some aspects, malonate semialdehyde can be converted to acetyl-CoA by a malonate semialdehyde dehydrogenase. In some aspects, the modified yeast comprises one or more malonate semialdehyde dehydrogenases including, but not limited to, enzymes with EC number 1.2.1.18 or EC number 1.2.1.27, such as those listed in Table 1. In some aspects, the malonate semialdehyde dehydrogenase (bauC) is from Pseudomonas aeruginosa. In some aspects, the malonate semialdehyde dehydrogenase (Ald6) is from Candida albicans. In some aspects, the malonate semialdehyde dehydrogenase (iolA) is from Lysteria monocytogenes. In some aspects, the malonate semialdehyde dehydrogenase (dddC) is from Halomonas sp. HTNK1.

TABLE 1
Candidates for conversion of malonate
semialdehyde to acetyl-CoA.

| | EC | | |
|---|---|---|---|
| Activity | Number | Gene | Organism |
| Malonate semialdehyde | 1.2.1.18 | bauC | Pseudomonas |
| dehydrogenase | | | aeruginosa |
| Malonate semialdehyde | 1.2.1.18 | Ald6 | Candida albicans |
| dehydrogenase | | | |
| Malonate semialdehyde | 1.2.1.27 | iolA | Lysteria |
| dehydrogenase | | | monocytogenes |
| Malonate semialdehyde | — | dddC | Halomonas sp. |
| dehydrogenase | | | HTNK1 |

In some aspects, malonate semialdehyde can be converted to acetyl-CoA by sequential reactions of (i) a malonyl-CoA reductase and/or a 2-keto acid decarboxylase, and (ii) a malonyl-CoA decarboxylase. In some aspects, the malonyl-CoA reductase and/or a 2-keto acid decarboxylase catalyzes the conversion of malonate semialdehyde into malonyl-CoA. In some aspects, the malonyl-CoA decarboxylase catalyzes the production of acetyl-CoA from malonyl-CoA. In some aspects, the modified yeast comprises one or more malonyl-CoA reductases and/or 2-keto acid decarboxylases including, but not limited to, enzymes with EC number 1.1.1.298, such as those listed in Table 2. In some aspects, the modified yeast comprises one or more malonyl-CoA decarboxylases including, but not limited to, enzymes with EC number 4.1.1.9, such as those listed in Table 2. In some aspects, the malonyl-CoA reductase (mcr) is from Chloroflexus aurantiacus. In some aspects, the 2-keto acid decarboxylase (kivD) is from Lactococcus lactis. In some aspects, the 2-keto acid decarboxylase (kdcA) is from Lactococcus lactis. In some aspects, the 2-keto acid decarboxylase (ARO10) is from Saccharomyces cerevisiae. In some aspects, the malonyl-CoA decarboxylase (MatA) is from Rhizobium trifolii. In some aspects, the malonyl-CoA decarboxylase (MLYCD) is from Homo sapiens.

TABLE 2
Candidates for conversion of malonate semialdehyde
to acetyl-CoA via a malonyl-CoA intermediate.

| | EC | | |
|---|---|---|---|
| Activity | Number | Gene | Organism |
| Malonyl-CoA reductase | 1.1.1.298 | mcr | Chloroflexus |
| | | | aurantiacus |
| 2-keto acid decarboxylase | — | kivD | Lactococcus lactis |
| 2-keto acid decarboxylase | — | kdcA | Lactococcus lactis |
| 2-keto acid decarboxylase | — | ARO10 | Saccharomyces |
| | | | cerevisiae |
| Malonyl-CoA | — | MatA | Rhizobium trifolii |
| decarboxylase | | | |
| Malonyl-CoA | 4.1.1.9 | MLYCD | Homo sapiens |
| decarboxylase | | | |

In some aspects, malonate semialdehyde can be converted to 3HP by a 3-hydroxypropionic acid dehydrogenase. In some aspects, the modified yeast comprises one or more 3-hydroxypropionic acid dehydrogenases including, but not limited to, enzymes with EC number 1.1.1.298 or EC number 1.1.1.381, such as those listed in Table 3. In some aspects, the 3-hydroxypropionic acid dehydrogenase (ydfg) is from Escherichia coli. In some aspects, the 3-hydroxypropionic acid dehydrogenase (mcr-1) is from Chloroflexus aurantiacus. In some aspects, the 3-hydroxypropionic acid dehydrogenase (Ydf1) is from Saccharomyces cerevisiae. In some aspects, the 3-hydroxypropionic acid dehydrogenase (Hpd1) is from Candida albicans.

TABLE 3
Candidates for conversion of malonate semialdehyde
to 3-hydroxypropionic acid.

| | EC | | |
|---|---|---|---|
| Activity | Number | Gene | Organism |
| 3-hydroxypropionic acid | 1.1.1.298 | ydfg | Escherichia coli |
| dehydrogenase | | | |
| 3-hydroxypropionic acid | — | mcr-1 | Chloroflexus |
| dehydrogenase | | | aurantiacus |
| 3-hydroxypropionic acid | 1.1.1.381 | Ydf1 | Saccharomyces |
| dehydrogenase | | | cerevisiae |
| 3-hydroxypropionic acid | — | Hpd1 | Candida albicans |
| dehydrogenase | | | |

In some aspects, 3HP can be converted to propionyl-CoA by the sequential reactions of (i) a 3-hydroxypropionyl-CoA transferase, a 3-hydroxypropionyl-CoA ligase, or a 3-hydroxypropionyl-CoA synthase; (ii) a 3-hydroxypropionyl-CoA dehydratase; and (iii) an acrylyl-CoA reductase.

In some aspects, the modified yeast comprises one or more 3-hydroxypropionyl-CoA transferases, 3-hydroxypropionyl-CoA ligases, and/or 3-hydroxypropionyl-CoA synthases including, but not limited to, enzymes with EC number 2.8.3.1, EC number 6.2.1.17, or EC number 6.2.1.36, such as those listed in Table 4. In some aspects, the 3-hydroxypropionyl-CoA transferase (pct) is from Cupriavidus necator, Clostridium propionicum, or Megasphaera elsdenii. In some aspects, the 3-hydroxypropionyl-CoA ligase (prpE) is from Salmonella enterica or Escherichia coli. In some aspects, the 3-hydroxypropionyl-CoA ligase (Nmar 1309) is from Nitrosopumilus maritimus. In some aspects, the 3-hydroxypropionyl-CoA synthase (Msed 1456) is from Metallosphaera sedula. In some aspects, the 3-hydroxypropionyl-CoA synthase (Stk 07830) is from Sulfolobus tokodaii.

In some aspects, the 3-hydroxypropionyl-CoA transferase transfers the coenzyme-A from acetyl-CoA to 3-hydroxypropionate generating acetate. The coenzyme is recycled by two sequential reactions wherein acetate is converted to acetate-P by an acetate kinase and acetate-P is converted to acetyl-CoA by a phosphate acetyltransferase. Acetate kinases and phosphate acetyltransferases include, but are not limited to, enzymes with EC number 2.7.2.1 and EC number 2.3.1.8, respectively. In some aspects, the acetate kinase is from Corynebacterium glutamicum or Escherichia coli. In some aspects, the acetate kinase is from Escherichia coli (ackA). In some aspects, the phosphate acetyltransferase is from Escherichia coli or Corynebacterium glutamicum. In some aspects, the phosphate acetyltransferase is from Corynebacterium glutamicum (pta). In some aspects, the phosphate acetyltransferase is from Corynebacterium glutamicum and the acetate kinase is from Escherichia coli.

In some aspects, the modified yeast comprises one or more 3-hydroxypropionyl-CoA dehydratases including, but not limited to, enzymes with EC number 4.2.1.116, EC number 4.2.1.55, EC number 4.2.1.150, or EC number 4.2.1.17, such as those listed in Table 4. In some aspects, the 3-hydroxypropionyl-CoA dehydratase (hpcd) is from Metallosphaera sedula, Bacillus sp., or Sporanaerobacter acetigenes. In some aspects, the 3-hydroxypropionyl-CoA dehydratase is from Ruegeria pomeroyi. In some aspects, the 3-hydroxypropionyl-CoA dehydratase (St1516) is from Sulfolobus tokodaii. In some aspects, the 3-hydroxypropionyl-CoA dehydratase (Nmar_1308) is from Nitrosopumilus maritimus. In some aspects, the 3-hydroxypropionyl-CoA dehydratase (Hpcd) is from Chloroflexus aurantiacus. In some aspects, the 3-hydroxypropionyl-CoA dehydratase (Crt) is from Clostridium acetobutylicum or Clostridium pasteuranum. In some aspects, the 3-hydroxypropionyl-CoA dehydratase is from Clostridium pasteuranum. In some aspects, the 3-hydroxypropionyl-CoA dehydratase (Mels_1449) is from Megasphaera elsdenii. In some aspects, the 3-hydroxypropionyl-CoA dehydratase (Aflv_0566) is from Anoxybacillus flavithermus.

In some aspects, the modified yeast comprises one or more acrylyl-CoA reductases including, but not limited to, enzymes with EC number 1.3.1.84 or EC number 1.3.1.95, such as those listed in Table 4. In some aspects, the acrylyl-CoA reductase (acuI) is from Ruegeria pomeroyi, Escherichia coli, or Rhodobacter sphaeroides. In some aspects, the acrylyl-CoA reductase (pcdh) is from Clostridium propionicum. In some aspects, the acrylyl-CoA reductase (acuI) is from Alcaligenes faecalis. In some aspects, the acrylyl-CoA reductase (Acr) is from Sulfolobus tokodaii. In some aspects, the acrylyl-CoA reductase (acuI) is from Escherichia coli. In some aspects, the acrylyl-CoA reductase (Acr) is from Metallosphaera sedula. In some aspects, the acrylyl-CoA reductase (Nmar_1565) is from Nitrosopumilus maritimus.

In some aspects, the 3-hydroxypropionyl-CoA transferase (pct) is from Clostridium propionicum, the 3-hydroxypropionyl-CoA dehydratase (hpcd) is from Sporanaerobacter acetigenes and/or Metallosphaera sedula, and the acrylyl-CoA reductase (acr) is from Ruegeria pomeroyi.

TABLE 4
Candidates for conversion of 3-hydroxypropionic
acid to propionyl-CoA.

| | EC | | |
|---|---|---|---|
| Activity | Number | Gene | Organism |
| Propionyl-CoA | 2.8.3.1 | pct | Cupriavidus necator |
| transferase | | | |
| Propionyl-CoA | 2.8.3.1 | pct | Clostridium |
| transferase | | | propionicum |
| Propionyl-CoA | 2.8.3.1 | pct | Megasphaera |
| transferase | | | elsdenii |
| Propionyl-CoA ligase | 6.2.1.17 | prpE | Salmonella enterica |
| Propionyl-CoA ligase | 6.2.1.17 | prpE | Escherichia coli |
| CoA ligase | — | Nmar_1309 | Nitrosopumilus |
| | | | maritimus |
| 3-hydroxypropionyl- | 6.2.1.36 | Msed_1456 | Metallosphaera |
| coenzyme A synthetase | | | sedula |
| 3-hydroxypropionyl- | 6.2.1.36 | Stk_07830 | Sulfolobus tokodaii |
| coenzyme A synthetase | | | |
| 3-hydroxypropionyl- | 4.2.1.116 | hpcd | Metallosphaera |
| coenzyme A | | | sedula |
| dehydratase | | | |
| Enoyl-CoA hydratase | — | hpcd | Bacillus sp. |
| Enoyl-CoA hydratase | — | hpcd | Sporanaerobacter |
| | | | acetigenes |
| Enoyl-CoA hydratase | — | — | Ruegeria pomeroyi |
| 3-hydroxypropionyl- | 4.2.1.116 | St1516 | Sulfolobus tokodaii |
| coenzyme A | | | |
| dehydratase | | | |
| Enoyl-CoA hydratase | 4.2.1.116 | Nmar_1308 | Nitrosopumilus |
| | | | maritimus |
| 3-hydroxypropionyl- | 4.2.1.116 | Hpcd | Chloroflexus |
| coenzyme A | | | aurantiacus |
| dehydratase | | | |
| Enoyl-CoA hydratase | 4.2.1.55 | Crt | Clostridium |
| | | | acetobutylicum |
| Enoyl-CoA hydratase | 4.2.1.55 | — | Clostridium |
| | | | pasteuranum |
| Enoyl-CoA hydratase | 4.2.1.150 | Crt | Clostridium |
| | | | pasteuranum |
| 3-hydroxybutyryl-CoA | 4.2.1.55 | Mels_1449 | Megasphaera |
| dehydratase | | | elsdenii |
| Enoyl-CoA hydratase | 4.2.1.17 | Aflv_0566 | Anoxybacillus |
| | | | flavithermus |
| Acrylyl-CoA reductase | 1.3.1.84 | acuI | Ruegeria pomeroyi |
| Acrylyl-CoA reductase | 1.3.1.84 | acuI | Escherichia coli |
| Acrylyl-CoA reductase | 1.3.1.84 | acuI | Rhodobacter |
| | | | sphaeroides |
| Acrylyl-CoA reductase | 1.3.1.95 | pcdh | Clostridium |
| | | | propionicum |
| Acrylyl-CoA reductase | 1.3.1.95 | acuI | Alcaligenes faecalis |
| Acrylyl-CoA reductase | 1.3.1.84 | Acr | Sulfolobus tokodaii |
| Acrylyl-CoA reductase | 1.3.1.84 | acuI | Escherichia coli |
| Acrylyl-CoA reductase | 1.3.1.84 | Acr | Metallosphaera |
| | | | sedula |
| Acrylyl-CoA reductase | — | Nmar_1565 | Nitrosopumilus |
| | | | maritimus |

In some aspects, 3HP can be converted to propionyl-CoA by a trifunctional propionyl-CoA synthase (PCS). In some aspects, the modified yeast comprises one or more propionyl-CoA synthases including, but not limited to, enzymes with EC number 6.2.1.17, such as those listed in Table 5. In some aspects, the propionyl-CoA synthase (pcs) is from Chlorojlexus aurantiacus, Chlorojlexus aggregans, Roseijlexus castenholzii, Natronococcus occultus, Halioglobus japonicus, or Erythrobacter sp. NAP1.

TABLE 5
Candidates for conversion of 3-hydroxypropionic
acid to propionyl-CoA.

| | EC | | |
|---|---|---|---|
| Activity | Number | Gene | Organism |
| Propionyl-CoA synthase | 6.2.1.17 | pcs | Chloroflexus |
| | | | aurantiacus |
| Propionyl-CoA synthase | 6.2.1.17 | pcs | Chloroflexus aggregans |
| Propionyl-CoA synthase | 6.2.1.17 | pcs | Roseiflexus castenholzii |
| Propionyl-CoA synthase | 6.2.1.17 | pcs | Natronococcus occultus |
| Propionyl-CoA synthase | 6.2.1.17 | pcs | Halioglobus japonicus |
| Propionyl-CoA synthase | 6.2.1.17 | pcs | Erythrobacter sp. NAP1 |

In some aspects, the modified yeast comprises: (i) at least one nucleic acid molecule encoding a polypeptide that catalyzes the production of 3-ketovaleryl-CoA from propionyl-CoA and acetyl-CoA; (ii) at least one nucleic acid molecule encoding a polypeptide that catalyzes the production of 3-oxovalerate from 3-ketovaleryl-CoA; and (iii) at least one nucleic acid molecule encoding a polypeptide that catalyzes the production of 2-butanone from 3-oxovalerate.

In some aspects, propionyl-CoA and acetyl-CoA together can be converted to 3-ketovaleryl-CoA by a β-ketothiolase or an acetyl-CoA acetyltransferase. In some aspects, the modified yeast comprises one or more β-ketothiolases or acetyl-CoA acetyltransferases including, but not limited to, enzymes with EC number 2.3.1.16 or EC number 2.3.1.9, such as those listed in Table 6. In some aspects, the β-ketothiolase (phaA) is from Acinetobacter sp. RA384. In some aspects, the β-ketothiolase (BktB) is from Cupriviadus necator. In some aspects, the β-ketothiolase (BktC) is from Cupriviadus necator. In some aspects, the β-ketothiolase (BktB) is from Cupriavidus taiwanensis. In some aspects, the β-ketothiolase (POT1) is from Saccharomyces cerevisiae. In some aspects, the acetyl-CoA acetyltransferase (phaA) is from Cupriavidus necator. In some aspects, the acetyl-CoA acetyltransferase (thlA) is from Clostridium acetobutylicum. In some aspects, the acetyl-CoA acetyltransferase (thlB) is from Clostridium acetobutylicum. In some aspects, the acetyl-CoA acetyltransferase (phaA) is from Zoogloea ramigera. In some aspects, the acetyl-CoA acetyltransferase (atoB) is from Escherichia coli. In some aspects, the acetyl-CoA acetyltransferase (ERG10) is from Saccharomyces cerevisiae.

TABLE 6
Candidates for conversion of propionyl-
CoA and acetyl-CoA to 3-ketovaleryl-CoA.

| | EC | | |
|---|---|---|---|
| Activity | Number | Gene | Organism |
| β-ketothiolase | 2.3.1.16 | phaA | Acinetobacter sp. |
| | | | RA3849 |
| β-ketothiolase | 2.3.1.16 | BktB | Cupriviadus necator |
| β-ketothiolase | 2.3.1.16 | BktC | Cupriviadus necator |
| β-ketothiolase | 2.3.1.16 | BktB | Cupriavidus taiwanensis |
| β-ketothiolase | 2.3.1.16 | POT1 | Saccharomyces |
| | | | cerevisiae |
| Acetyl-CoA | 2.3.1.9 | phaA | Cupriavidus necator |
| acetyltransferase | | | |
| Acetyl-CoA | 2.3.1.9 | thlA | Clostridium |
| acetyltransferase | | | acetobutylicum |
| Acetyl-CoA | 2.3.1.9 | thlB | Clostridium |
| acetyltransferase | | | acetobutylicum |
| Acetyl-CoA | 2.3.1.9 | phaA | Zoogloea ramigera |
| acetyltransferase | | | |
| Acetyl-CoA | 2.3.1.9 | atoB | Escherichia coli |
| acetyltransferase | | | |
| Acetyl-CoA | 2.3.1.9 | ERG10 | Saccharomyces |
| acetyltransferase | | | cerevisiae |

In some aspects, 3-ketovaleryl-CoA can be converted to 3-ketovalerate (also known as 3-oxovalerate) by a 3-ketovaleryl-CoA transferase or a 3-ketovaleryl-CoA hydrolase. In some aspects, the modified yeast comprises one or more 3-ketovaleryl-CoA transferases or 3-ketovaleryl-CoA hydrolases selected from succinyl-CoA:3-ketoacid-CoA transferases, acetate-CoA transferases, butyrate-acetoacetate-CoA transferases, and acetoacetyl-CoA:acetyl-CoA transferases, including, but not limited to, enzymes with EC number 2.8.3.5, EC number 2.8.3.8, or EC number 2.8.3.9, such as those listed in Table 7. In some aspects, the succinyl-CoA:3-ketoacid-CoA transferase (ScoA) is from Bacillus subtilis. In some aspects, the succinyl-CoA:3-ketoacid-CoA transferase (ScoB) is from Bacillus subtilis. In some aspects, the acetate-CoA transferase (atoA) is from Escherichia coli. In some aspects, the acetate-CoA transferase (atoD) is from Escherichia coli. In some aspects, the butyrate-acetoacetate-CoA transferase (ctfA) is from Clostridium acetobutylicum. In some aspects, the butyrate-acetoacetate-CoA transferase (cam) is from Clostridium acetobutylicum. In some aspects, the butyrate-acetoacetate-CoA transferase (ctfA) is from Clostridium saccharoperbutylacetonicum. In some aspects, the butyrate-acetoacetate-CoA transferase (ctfB) is from Clostridium saccharoperbutylacetonicum. In some aspects, the acetoacetyl-CoA:acetyl-CoA transferase (ctfA) is from Escherichia coli. In some aspects, the acetoacetyl-CoA:acetyl-CoA transferase (cam) is from Escherichia coli. In some aspects, the acetate CoA-transferase (ydiF) is from Escherichia coli.

In some aspects, transferases transfer the coenzyme-A from 3-ketovaleryl-CoA to acetate generating acetyl-CoA. Acetate is recycled by two sequential reactions where acetyl-CoA is converted to acetyl-P by a phosphate acetyltransferase and acetyl-P is converted to acetate by an acetate kinase. Acetate kinases and phosphate acetyltransferases include, but are not limited to, enzymes with EC number 2.7.2.1 and EC number 2.3.1.8, respectively. In some aspects, the acetate kinase is from Corynebacterium glutamicum or Escherichia coli. In some aspects, the acetate kinase is from Escherichia coli (ackA). In some aspects, the phosphate acetyltransferase is from Escherichia coli or Corynebacterium glutamicum. In some aspects, the phosphate acetyltransferase is from Corynebacterium glutamicum (pta). In some aspects, the phosphate acetyltransferase is from Corynebacterium glutamicum and the acetate kinase is from Escherichia coli.

TABLE 7
Candidates for conversion of 3-ketovaleryl-
CoA to 3-ketovalerate (3-oxovalerate).

| | EC | | |
|---|---|---|---|
| Activity | Number | Gene | Organism |
| Succinyl-CoA:3-ketoacid- | 2.8.3.5 | ScoA | Bacillus subtilis |
| CoA transferase | | | |
| Succinyl-CoA:3-ketoacid- | 2.8.3.5 | ScoB | Bacillus subtilis |
| CoA transferase | | | |
| Acetate CoA-transferase | 2.8.3.8 | atoA | Escherichia coli |
| Acetate CoA-transferase | 2.8.3.8 | atoD | Escherichia coli |
| Butyrate-acetoacetate | 2.8.3.9 | ctfA | Clostridium acetobutylicum |
| CoA-transferase | | | |
| Butyrate-acetoacetate | 2.8.3.9 | ctfB | Clostridium acetobutylicum |
| CoA-transferase | | | |
| Butyrate-acetoacetate | 2.8.3.9 | ctfA | Clostridium |
| CoA-transferase | | | saccharoperbutyl- |
| | | | acetonicum |
| Butyrate-acetoacetate | 2.8.3.9 | ctfB | Clostridium |
| CoA-transferase | | | saccharoperbutyl- |
| | | | acetonicum |
| Acetoacetyl-CoA:acetyl- | 2.8.3.9 | ctfA | Escherichia coli |
| CoA transferase | | | |
| Acetoacetyl-CoA:acetyl- | 2.8.3.9 | ctfB | Escherichia coli |
| CoA transferase | | | |
| Acetate CoA-transferase | 2.8.3.8 | ydiF | Escherichia coli |

In some aspects, 3-ketovalerate (also known as 3-oxovalerate), which is structurally similar to acetoacetate, can be converted to butanone by an acetoacetate decarboxylase. In some aspects, the modified yeast comprises one or more enzymes with acetoacetate decarboxylase activity, including, but not limited to, enzymes with EC number 4.1.1.4, such as those listed in Table 8. In some aspects, the acetoacetate decarboxylase (adc) is from Clostridium acetobutylicum. In some aspects, the acetoacetate decarboxylase (adc) is from Clostridium saccharoperbutylacetonicum. In some aspects, the acetoacetate decarboxylase (adc) is from Clostridium beijerinkii. In some aspects, the acetoacetate decarboxylase (adc) is from Clostridium pasteuranum. In some aspects, the acetoacetate decarboxylase (adc) is from Pseudomonas putida.

TABLE 8
Candidates for conversion of 3-ketovalerate
(3-oxovalerate) to butanone.

| | EC | | |
|---|---|---|---|
| Activity | Number | Gene | Organism |
| Acetoacetate | 4.1.1.4 | adc | Clostridium acetobutylicum |
| decarboxylase | | | |
| Acetoacetate | 4.1.1.4 | adc | Clostridium |
| decarboxylase | | | saccharoperbutylacetonicum |
| Acetoacetate | 4.1.1.4 | adc | Clostridium beijerinkii |
| decarboxylase | | | |
| Acetoacetate | 4.1.1.4 | adc | Clostridium pasteuranum |
| decarboxylase | | | |
| Acetoacetate | 4.1.1.4 | adc | Pseudomonas putida |
| decarboxylase | | | |

In some aspects, the enzymes used to convert propionyl-CoA and acetyl-CoA to butanone are (i) a β-ketothiolase (BktB) from Cupriavidus necator and/or a β-ketothiolase (phaA) from Acinetobacter sp., (ii) a CoA transferase (atoAD) from Escherichia coli and/or a CoA transferase (ctfAB) from Clostridium acetobutylicum, and (iii) an acetate decarboxylase (adc) from Clostridium acetobutylicum or Pseudomonas putida. Advantageously, in some aspects, the enzymes convert propionyl-CoA and acetyl-CoA into butanone without formation of significant levels of undesired by-products such as acetone, thereby avoiding undesirable decreases in yield.

In some aspects, the modified yeast comprises: (i) at least one nucleic acid molecule encoding a polypeptide that catalyzes the production of 2-methylacetoacetyl-CoA from propionyl-CoA and acetyl-CoA; (ii) at least one nucleic acid molecule encoding a polypeptide that catalyzes the production of 2-methylacetoacetate from 2-methylacetoacetyl-CoA; and (iii) at least one nucleic acid molecule encoding a polypeptide that catalyzes the production of 2-butanone from 2-methylacetoacetate.

In some aspects, propionyl-CoA and acetyl-CoA together can be converted to 2-methylacetoacetyl-CoA by a 2-methylacetoacetyl-CoA thiolase. In some aspects, 2-methylacetoacetyl-CoA can be converted to 2-methylacetoacetate by a CoA hydrolase or a CoA-transferase. In some aspects, the CoA hydrolase is an acetyl-CoA hydrolase. In some aspects, the CoA-transferase is an acetyl-CoA acetyltransferase or a succinyl-CoA:3-ketoacid-CoA transferase. In some aspects, the modified yeast comprises one or more CoA hydrolases or CoA-transferases including, but not limited to, enzymes with EC number 2.3.1.9, EC number 2.8.3.5, or EC number 3.1.2.1, such as those listed in Table 9. In some aspects, the acetyl-CoA acetyltransferase (Act1) is from Homo sapiens. In some aspects, the succinyl-CoA:3-ketoacid-CoA transferase (ScoA) is from Bacillus subtilis. In some aspects, the succinyl-CoA:3-ketoacid-CoA transferase (ScoB) is from Bacillus subtilis. In some aspects, the acetyl-CoA hydrolase (Ach1) is from Saccharomyces cerevisiae.

In some aspects, 2-methylacetoacetate can be converted to butanone by a 2-methylacetoacetate decarboxylase. In some aspects, the modified yeast comprises one or more 2-methylacetoacetate decarboxylases including, but not limited to, enzymes with EC number 4.1.1.5, such as those listed in Table 9. In some aspects, the 2-methylacetoacetate decarboxylase is an A-acetolactate decarboxylase. In some aspects, the A-acetolactate decarboxylase (ALDC) is from Acetobacter aceti. In some aspects, the A-acetolactate decarboxylase (Aldc) is from Enterobacter aerogenes. In some aspects, the A-acetolactate decarboxylase (budA) is from Rauoltella terrigena.

TABLE 9
Candidates for conversion of propionyl-
CoA and acetyl-CoA to butanone.

| | EC | | |
|---|---|---|---|
| Activity | Number | Gene | Organism |
| Acetyl-CoA | 2.3.1.9 | Act1 | Homo sapiens |
| acetyltransferase | | | |
| Succinyl-CoA:3-ketoacid- | 2.8.3.5 | ScoA | Bacillus subtilis |
| CoA transferase | | | |
| Succinyl-CoA:3-ketoacid- | 2.8.3.5 | ScoB | Bacillus subtilis |
| CoA transferase | | | |
| Acetyl-CoA hydrolase | 3.1.2.1 | Ach1 | Saccharomyces |
| | | | cerevisiae |
| A-acetolactate | 4.1.1.5 | ALDC | Acetobacter aceti |
| decarboxylase | | | |
| A-acetolactate | 4.1.1.5 | Aldc | Enterobacter aerogenes |
| decarboxylase | | | |
| A-acetolactate | — | budA | Rauoltella terrigena |
| decarboxylase | | | |

In some aspects, butanone can be converted into 2-butanol by an alcohol dehydrogenase (e.g., a 2-butanol dehydrogenase) or a MEK reductase. In some aspects, the alcohol dehydrogenase is NAD-dependent. In some aspects, the alcohol dehydrogenase is NADP-dependent.

In some aspects, the modified yeast comprises one or more alcohol dehydrogenases including, but not limited to, enzymes with EC number 1.1.1.1, EC number 1.1.1.2, EC number 1.1.1.80, or EC number 1.1.1.-, such as those listed in Table 10. In some aspects, NAD-dependent enzymes are known as EC number 1.1.1.1. In some aspects, NADP-dependent enzymes are known as EC number 1.1.1.2. In some aspects, the 2-butanol dehydrogenase (sadh) is from Rhodococcus ruber. In some aspects, the 2-butanol dehydrogenase (adhA) is from Pyrococcus furious. In some aspects, the 2-butanol dehydrogenase (adh) is from Clostridium beijerinckii. In some aspects, the 2-butanol dehydrogenase (adh) is from Thermoanaerobacter brockii. In some aspects, the 2-butanol dehydrogenase (yqhD) is from Escherichia coli. In some aspects, the 2-butanol dehydrogenase (chnA) is from Acinetobacter sp.

TABLE 10
Candidates for conversion of butanone to 2-butanol.

| | EC | | |
|---|---|---|---|
| Activity | Number | Gene | Organism |
| 2-butanol dehydrogenase | 1.1.1.1 | sadh | Rhodococcus ruber |
| 2-butanol dehydrogenase | 1.1.1.2 | adhA | Pyrococcus furious |
| 2-butanol dehydrogenase | 1.1.1.80 | adh | Clostridium beijerinckii |
| 2-butanol dehydrogenase | 1.1.1.80 | adh | Thermoanaerobacter |
| | | | brockii |
| 2-butanol dehydrogenase | 1.1.1.— | yqhD | Escherichia coli |
| 2-butanol dehydrogenase | — | chnA | Acinetobacter sp. |

### Pathways for Production of Methyl Propionate

In another pathway, methyl propionate is produced from butanone by a Baeyer-Villiger monooxygenases including, but not limited to, enzymes with EC number 1.14.13.-. In an embodiment, the Baeyer-Villiger monooxygenase is from Acinetobacter calcoaceticus, Rhodococcus jostii, and/or Xanthobacter flavus.

### Pathways for Co-Production of 1-Propanol and Butanone

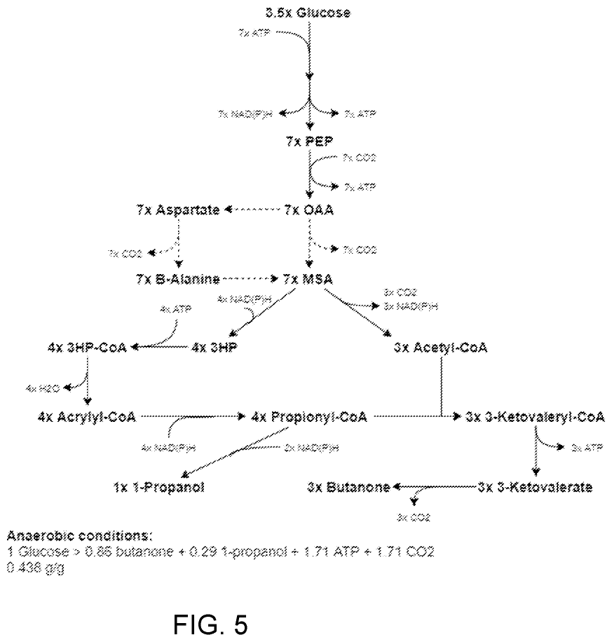

In another pathway, 1-propanol and butanone are co-produced from malonate semialdehyde (MSA) as shown FIG. 5. Metabolic pathways for the co-production of 1-propanol with butanone include pathways that produce 1-propanol and butanone from intermediates including, but not limited to, malonate semialdehyde, 3-hydroxypropionic acid (3HP), 3-hydroxypropionyl-coenzyme A (3HP-CoA), acrylyl-CoA, propionyl-CoA, acetyl-CoA, 3-ketovaleryl-CoA, and 3-ketovalerate. In the pathways for production of butanone discussed herein, a portion of the produced propionyl-CoA is used to produce butanone and a portion is used to produce 1-propanol.

In some aspects, propionyl-CoA can be converted to 1-propanol by a bifunctional alcohol/aldehyde dehydrogenase. In some aspects, the modified yeast comprises one or more bifunctional alcohol/aldehyde dehydrogenases including, but not limited to, enzymes with EC number 1.1.1.1, EC number 1.2.1.4, or EC number 1.2.1.5, such as those listed in Table 11. In some aspects, the alcohol/aldehyde dehydrogenase (adhe) is from Clostridium acetobutylicum. In some aspects, the alcohol/aldehyde dehydrogenase (adhe) is from Clostridium beijerinckii. In some aspects, the alcohol/aldehyde dehydrogenase (adhe) is from Clostridium typhimurium. In some aspects, the alcohol/aldehyde dehydrogenase (adhe) is from Clostridium arbusti. In some aspects, the alcohol/aldehyde dehydrogenase (adhE) is from Escherichia coli. In some aspects, the alcohol/aldehyde dehydrogenase (adhP) is from Escherichia coli. In some aspects, the alcohol/aldehyde dehydrogenase (bdhB) is from Clostridium acetobutylicum. In some aspects, the alcohol/aldehyde dehydrogenase (Adh2) is from Saccharomyces cerevisiae. In some aspects, the alcohol/aldehyde dehydrogenase (adhE) is from Clostridium roseum. In some aspects, the alcohol/aldehyde dehydrogenase (adhA) is from Thermoanaerobacterium saccharolyticum. In some aspects, the alcohol/aldehyde dehydrogenase (Ald6) is from Saccharomyces cerevisiae. In some aspects, the alcohol/aldehyde dehydrogenase (Aldh3A1) is from Homo sapiens.

TABLE 11
Candidates for direct conversion of propionyl-CoA to 1-propanol.

| | EC | | |
|---|---|---|---|
| Activity | Number | Gene | Organism |
| Aldehyde/alcohol | — | adhe | Clostridium |
| dehydrogenase | | | acetobutylicum |
| Aldehyde/alcohol | — | adhe | Clostridium beijerinckii |
| dehydrogenase | | | |
| Aldehyde/alcohol | — | adhe | Clostridium |
| dehydrogenase | | | typhimurium |
| Aldehyde/alcohol | — | adhe | Clostridium arbusti |
| dehydrogenase | | | |
| Aldehyde/alcohol | 1.1.1.1 | adhE | Escherichia coli |
| dehydrogenase | | | |
| Aldehyde/alcohol | 1.1.1.1 | adhP | Escherichia coli |
| dehydrogenase | | | |
| Aldehyde/alcohol | 1.1.1.1 | bdhB | Clostridium |
| dehydrogenase | | | acetobutylicum |
| Aldehyde/alcohol | 1.1.1.1 | Adh2 | Saccharomyces |
| dehydrogenase | | | cerevisiae |
| Aldehyde/alcohol | — | adhE | Clostridium roseum |
| dehydrogenase | | | |
| Aldehyde/alcohol | — | adhA | Thermoanaerobacterium |
| dehydrogenase | | | saccharolyticum |
| Aldehyde/alcohol | 1.2.1.4 | Ald6 | Saccharomyces |
| dehydrogenase | | | cerevisiae |
| Aldehyde/alcohol | 1.2.1.5 | Aldh3A1 | Homo sapiens |
| dehydrogenase | | | |

In some aspects, propionyl-CoA can be converted to 1-propanol by sequential reactions of an aldehyde dehydrogenase (acetylating) and an alcohol dehydrogenase. In some aspects, the modified yeast comprises one or more aldehyde dehydrogenases (acetylating) including, but not limited to, enzymes with EC number 1.2.1.10, such as those listed in Table 12. In some aspects, the aldehyde dehydrogenases (acetylating) (mhpf) is from Escherichia coli. In some aspects, the aldehyde dehydrogenases (acetylating) (Mhpf) is from Escherichia coli. In some aspects, the aldehyde dehydrogenases (acetylating) (Mhpf) is from Escherichia coli. In some aspects, the aldehyde dehydrogenases (acetylating) (mhpf) is from Escherichia coli. In some aspects, the aldehyde dehydrogenases (acetylating) (Pdup) is from Escherichia coli. In some aspects, the aldehyde dehydrogenases (acetylating) (pdup) is from Escherichia coli. In some aspects, the aldehyde dehydrogenases (acetylating) (Pdup) is from Escherichia coli. In some aspects, the aldehyde dehydrogenases (acetylating) (aldH) is from Escherichia coli. In some aspects, the aldehyde dehydrogenases (acetylating) (ald) is from Escherichia coli. In some aspects, the modified yeast comprises one or more alcohol dehydrogenase including, but not limited to, enzymes with EC number 1.1.1.2 or EC number 1.2.1.87, such as those listed in Table 12. In some aspects, the alcohol dehydrogenase (alrA) is from Acinetobacter sp. In some aspects, the alcohol dehydrogenase (bdhI) is from Clostridium acetobutylicum. In some aspects, the alcohol dehydrogenase (bdhII) is from Clostridium acetobutylicum. In some aspects, the alcohol dehydrogenase (adhA) is from Clostridium glutamicum. In some aspects, the alcohol dehydrogenase (yqhD) is from Escherichia coli. In some aspects, the alcohol dehydrogenase (adhP) is from Escherichia coli. In some aspects, the alcohol dehydrogenase (PduQ) is from Propionibacterium freudenreichii. In some aspects, the alcohol dehydrogenase (ADH1) is from Saccharomyces cerevisiae. In some aspects, the alcohol dehydrogenase (ADH2) is from Saccharomyces cerevisiae. In some aspects, the alcohol dehydrogenase (ADH4) is from Saccharomyces cerevisiae. In some aspects, the alcohol dehydrogenase (ADH6) is from Saccharomyces cerevisiae. In some aspects, the alcohol dehydrogenase (PduQ) is from Salmonella enterica. In some aspects, the alcohol dehydrogenase (Adh) is from Sulfolobus tokodaii. In some aspects, the aldehyde dehydrogenase (acetylating) (PduP) is from Salmonella enterica and the alcohol dehydrogenase (ADH1) is from Saccharomyces cerevisiae.

TABLE 12
Candidates for conversion of propionyl-CoA to propionaldehyde
and for conversion of propionaldehyde to 1-propanol.

| | EC | | |
|---|---|---|---|
| Activity | Number | Gene | Organism |
| Aldehyde dehydrogenase | 1.2.1.10 | mhpf | Escherichia coli |
| (acetylating) | | | |
| Aldehyde dehydrogenase | 1.2.1.10 | Mhpf | Pseudomonas putida |
| (acetylating) | | | |
| Aldehyde dehydrogenase | 1.2.1.10 | Mhpf | Pseudomonas |
| (acetylating) | | | fluorescens |
| Aldehyde dehydrogenase | 1.2.1.10 | mhpf | Paraburkholderia |
| (acetylating) | | | xenovorans |
| Aldehyde dehydrogenase | — | Pdup | Salmonella enterica |
| (acetylating) | | | |
| Aldehyde dehydrogenase | — | pdup | Listeria monocytogenes |
| (acetylating) | | | |
| Aldehyde dehydrogenase | — | Pdup | Klebsiella pneumoniae |
| (acetylating) | | | |
| Aldehyde dehydrogenase | 1.2.1.10 | aldH | Acinetobacter sp. |
| (acetylating) | | | |
| Aldehyde dehydrogenase | 1.2.1.10 | ald | Clostridium beijerinckii |
| (acetylating) | | | |
| Alcohol dehydrogenase | 1.1.1.2 | alrA | Acinetobacter sp. |
| Alcohol dehydrogenase | 1.1.1.2 | bdhI | Clostridium |
| | | | acetobutylicum |
| Alcohol dehydrogenase | 1.1.1.2 | bdhII | Clostridium |
| | | | acetobutylicum |
| Alcohol dehydrogenase | 1.1.1.2 | adhA | Clostridium glutamicum |
| Alcohol dehydrogenase | 1.1.1.2 | yqhD | Escherichia coli |
| Alcohol dehydrogenase | 1.1.1.2 | adhP | Escherichia coli |
| Alcohol dehydrogenase | 1.1.1.2 | PduQ | Propionibacterium |
| | | | freudenreichii |
| Alcohol dehydrogenase | 1.1.1.2 | ADH1 | Saccharomyces |
| | | | cerevisiae |
| Alcohol dehydrogenase | 1.1.1.2 | ADH2 | Saccharomyces |
| | | | cerevisiae |
| Alcohol dehydrogenase | 1.1.1.2 | ADH4 | Saccharomyces |
| | | | cerevisiae |
| Alcohol dehydrogenase | 1.1.1.2 | ADH6 | Saccharomyces |
| | | | cerevisiae |
| Alcohol dehydrogenase | 1.1.1.2 | PduQ | Salmonella enterica |
| Alcohol dehydrogenase | 1.1.1.2 | Adh | Sulfolobus tokodaii |

Advantageously, the butanone and 1-propanol co-production pathway is redox neutral and ATP positive, resulting in a more efficient and higher yield production of the desired compounds. Furthermore, the balanced pathway has the potential to be performed under anaerobic conditions, which provides several fermentation process advantages when compared with an aerobic process with the same yield: anaerobic fermenters have reduced cost compared to aerobic fermentation, air compressors are expensive and represent cost increase, larger fermenters are possible for anaerobic processes so less number of fermenters needed compared to aerobic process based on the same product production capacity.

In some aspects, at least a portion of excess NAD (P)H produced by the modified yeast in the production of butanone is utilized to supply NAD(P)H in the production of 1-propanol. Without wishing to be bound by theory, it is believed that the redox balanced co-production of butanone and 1-propanol facilitates fermentation under anaerobic conditions without forming significant levels of undesired byproducts and thereby avoiding yield decrease for the desired products.

In some aspects, co-production of butanone and 1-propanol is carried out in an industrial ethanol-producing yeast strain. In some aspects, the industrial ethanol-producing yeast strain is engineered to co-produce butanone and 1-propanol under anaerobic fermentation condition wherein a portion of the carbon source is diverted to production of butanone and 1-propanol while continuing to produce ethanol. In some aspects, the industrial ethanol-producing yeast strain retains substantially all of its industrial ethanol yeast performance and robustness, thereby allow its use and successful implementation into existing industrial ethanol production operations.

### Modified Yeast

A modified yeast as provided herein may comprise:
one or more polynucleotides coding for enzymes in a pathway that catalyzes a conversion of a fermentable carbon source to succinyl-CoA,
one or more polynucleotides coding for enzymes in a pathway that catalyzes a conversion of a fermentable carbon source to 1,2-propanediol,
one or more polynucleotides coding for enzymes in a pathway that catalyzes a conversion of a fermentable carbon source to lactate,
one or more polynucleotides coding for enzymes in a pathway that catalyzes a conversion of a fermentable carbon source to β-alanine,
one or more polynucleotides coding for enzymes in a pathway that catalyzes a conversion of a fermentable carbon source to threonine,
one or more polynucleotides coding for enzymes in a pathway that catalyzes a conversion of a fermentable carbon source to citramalate,
one or more polynucleotides coding for enzymes in a pathway that catalyzes a conversion of fermentable carbon source to malonate semialdehyde,
one or more polynucleotides coding for enzymes in a pathway that catalyzes a conversion of succinyl-CoA to methylmalonyl-CoA,
one or more polynucleotides coding for enzymes in a pathway that catalyzes a conversion of threonine to 2-ketobutyrate (2-kB),
one or more polynucleotides coding for enzymes in a pathway that catalyzes a conversion of citramalate to 2-ketobutyrate (2-kB),
one or more polynucleotides coding for enzymes in a pathway that catalyzes a conversion of β-alanine to malonate semialdehyde,
one or more polynucleotides coding for enzymes in a pathway that catalyzes a conversion of malonate semialdehyde to 3-hydroxypropionate β-HP),
one or more polynucleotides coding for enzymes in a pathway that catalyzes a conversion of lactate to acrylyl-CoA,
one or more polynucleotides coding for enzymes in a pathway that catalyzes a conversion of β-alanine to acrylyl-CoA,
one or more polynucleotides coding for enzymes in a pathway that catalyzes a conversion of 3-HP to acrylyl-CoA,
one or more polynucleotides coding for enzymes in a pathway that catalyzes a conversion of methylmalonyl-CoA to propionyl-CoA,
one or more polynucleotides coding for enzymes in a pathway that catalyzes a conversion of 2-kB to propionyl-CoA,
one or more polynucleotides coding for enzymes in a pathway that catalyzes a conversion of acrylyl-CoA to propionyl-CoA,
one or more polynucleotides coding for enzymes in a pathway that catalyzes a conversion of propionyl-CoA to propionaldehyde,
one or more polynucleotides coding for enzymes in a pathway that catalyzes a conversion of 1,2-propanediol to propionaldehyde, and/or
one or more polynucleotides coding for enzymes in a pathway that catalyzes a conversion of propionaldehyde to 1-propanol.

A modified microorganism as provided herein may comprise:
one or more polynucleotides coding for enzymes in a pathway that catalyzes a conversion of fermentable carbon source to pyruvate,
one or more polynucleotides coding for enzymes in a pathway that catalyzes a conversion of fermentable carbon source to malonate semialdehyde (MSA),
one or more polynucleotides coding for enzymes in a pathway that catalyzes a conversion of pyruvate to acetyl-CoA,
one or more polynucleotides coding for enzymes in a pathway that catalyzes a conversion of MSA to acetyl-CoA;
one or more polynucleotides coding for enzymes in a pathway that catalyzes a conversion of acetyl-CoA to acetoacetyl-CoA,
one or more polynucleotides coding for enzymes in a pathway that catalyzes a conversion of acetyl-CoA to malonyl-CoA,
one or more polynucleotides coding for enzymes in a pathway that catalyzes a conversion of malonyl-CoA to acetoacetyl-CoA,
one or more polynucleotides coding for enzymes in a pathway that catalyzes a conversion of acetoacetyl-CoA to acetoacetate,
one or more polynucleotides coding for enzymes in a pathway that catalyzes a conversion of acetoacetyl-CoA to hydroxymethylglutaryl-CoA (HMG-CoA),
one or more polynucleotides coding for enzymes in a pathway that catalyzes a conversion of HMG-CoA to acetoacetate,
one or more polynucleotides coding for enzymes in a pathway that catalyzes a conversion of acetoacetate to acetone,
one or more polynucleotides coding for enzymes in a pathway that catalyzes a conversion of acetone to 2-propanol,
one or more polynucleotides coding for enzymes in a pathway that catalyzes a conversion of a fermentable carbon source to butyrate,
one or more polynucleotides coding for enzymes in a pathway that catalyzes a conversion of butyrate to propane,
one or more polynucleotides coding for enzymes in a pathway that catalyzes a conversion of propane to 2-propanol,
one or more polynucleotides coding for enzymes in a pathway that catalyzes a conversion of fermentable carbon source to 2-propanol,
one or more polynucleotides coding for enzymes in a pathway that catalyzes a conversion of 2-propanol to propene
one or more polynucleotides coding for enzymes in a pathway that catalyzes a conversion of a fermentable carbon source to butyryl-CoA,
one or more polynucleotides coding for enzymes in a pathway that catalyzes a conversion of butyrate to butanal,
one or more polynucleotides coding for enzymes in a pathway that catalyzes a conversion of butyryl-CoA to butanal, and/or
one or more polynucleotides coding for enzymes in a pathway that catalyzes a conversion of butanal to 1-butanol.

In some embodiments, the yeast is Saccharomyces cerevisiae, Kluyveromyces lactis or Pichia pastoris.

In some embodiments, the yeast is Saccharomyces cerevisiae and is an industrial ethanol producer yeast, i.e., a yeast strain already used in existing industrial ethanol fermentation processes and assets, wherein such industrial yeast has appropriate and distinguished robustness and fermentation performance to the production of ethanol.

In some embodiments, the yeast is Saccharomyces cerevisiae and is an industrial ethanol producer yeast already used in existing industrial ethanol fermentation processes and assets, wherein such processes and assets are based on sugar cane, sugar beets or corn as a raw material.

In some embodiments, the yeast is Saccharomyces cerevisiae and is an industrial ethanol producer yeast derived from or industrially used in already existing corn-based ethanol fermentation processes and assets.

In some embodiments, the yeast is additionally modified to comprise one or more tolerance mechanisms including, for example, tolerance to a produced molecule (e.g., 1-propanol, acetone, 2-propanol, propene, 1-butanol, 2-butanol, methyl ethyl ketone, and/or methyl propionate), and/or organic solvents. A yeast modified to comprise such a tolerance mechanism may provide a means to increase titers of fermentations and/or may control contamination in an industrial scale process.

Host cells are transformed or transfected with the above-described expression or cloning vectors for production of one or more enzymes as disclosed herein or with polynucleotides coding for one or more enzymes as disclosed herein and cultured in conventional nutrient media modified as appropriate for inducing promoters, selecting transformants, or amplifying the genes encoding the desired sequences.

Host cells containing desired nucleic acid sequences coding for the disclosed enzymes may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. In addition, any of the media described in Ham et al., Meth. Enz. 58: 44, (1979); Barnes et al., Anal. Biochem. 102: 255 (1980); U.S. Pat. Nos. 4,767,704; 4,657,866; 4,927,762; 4,560,655; or 5,122,469; WO 90/103430; WO 87/00195; or U.S. Pat. Re. No. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

### Methods for the Co-Production of Ethanol and a Co-Product

Ethanol and one or more co-products may be produced by contacting any of the genetically modified yeast provided herein with a fermentable carbon source. Such methods may preferably comprise contacting a fermentable carbon source with a yeast comprising one or more polynucleotides coding for enzymes in a pathway that catalyzes a conversion of the fermentable carbon source to any of the intermediates in the production of the co-product and one or more polynucleotides coding for enzymes in a pathway that catalyze a conversion of the one or more intermediates to the co-product in a fermentation media; and expressing the one or more polynucleotides coding for the enzymes in the pathway that catalyzes a conversion of the fermentable carbon source to the one or more intermediates in the production of the co-product and one or more polynucleotides coding for enzymes in a pathway that catalyze a conversion of the one or more intermediates to the co-product.

The fermentation products of the disclosure may be prepared by conventional processes for industrial sugar cane, sugar beets, or more preferably, corn ethanol production. In such processes, glucose and dextrose or another suitable carbon source can be derived from renewable grain sources through saccharification of starch-based feedstocks including grains such as corn, wheat, rye, barley, oats, rice, and mixtures thereof. Suitable carbon sources also include, but are not limited to, glucose, fructose, and sucrose, or mixtures of these with C5 sugars such as xylose and/or arabinose. The carbon source may also be derived from renewable sugar sources such as sugar cane, sugar beets, cassava, sweet sorghum, and mixtures thereof.

The fermentation media may additionally contain suitable minerals, salts, cofactors, buffers and other components suitable for the growth and maintenance of the cultures.

Fermentation processes such as corn ethanol production are typically performed in two stages: a yeast propagation phase and a fermentation phase. In the yeast propagation phase, yeast mass is increased to adequate quantities for the fermentation phase. Typically, the propagation phase is performed in sequential seed tanks. Appropriate culture media containing salts, nutrients and carbon sources (e.g., hydrolysate corn mash, sugarcane molasses or any other low-cost carbon source) are contacted with active dry yeast (ADY), yeast slurry or compressed yeast. Preferably, yeast propagation occurs under aerobic condition, but can also be done under anaerobic conditions. When an adequate yeast concentration is reached, the material is transferred to fermentation tanks to begin the fermentation phase. In the fermentation phase, the carbon source is converted to the main product such as ethanol and other by-products derived from yeast native metabolism. The fermentation phase of corn ethanol production uses the mash prepared from ground corn in a dry-grind or wet-milling process. Wet-milling processes involve fractionating the corn into different components where only the starch fraction enters into the fermentation process. Dry-grind processes involve grinding the corn kernels into meal and mixing the meal with water and enzymes. Generally, two different kinds of dry-grind processes are used. A commonly used process (the “conventional process”) involves grinding the starch-containing material and then liquefying gelatinized starch at a high temperature, typically using a bacterial alpha-amylase, followed by simultaneous saccharification and fermentation (SSF). Another well-known process, often referred to as a “raw starch hydrolysis” process (RSH process), includes grinding the starch-containing material and then simultaneously saccharifying and fermenting granular starch below the initial gelatinization temperature typically in the presence of an acid fungal alpha-amylase and a glucoamylase (see, e.g., U.S. Pat. No. 8,962,286).

In various embodiments, the fermentation runs at a temperature in the range of about 15° C. to about 60° C., preferably in a range between 28° C. to about 35° C. In various embodiments, the pH range for the fermentation is between pH 2.0 to pH 9.0. In some cases, the initial pH condition is pH 6.0 to pH 8.0. Fermentations can be performed under either aerobic or anaerobic conditions. Corn ethanol fermentation typically is conducted under anaerobic or microaerobic conditions. In some embodiments, air can be supplied during fermentation.

Suitable fermentation run times are in the range of about 24 to about 96 hours, such as about 36 hours to about 72 hours. Fermentation run time will vary based on the amount of yeast transferred from the propagation phase and the amount of starch enzyme during mash preparation and during the SSF process or RSH process. Once the carbon source is exhausted, the fermented mash is transferred to a downstream process (DPS) to purify the produced ethanol and other added cost by-products (e.g., dried distiller's grains with solubles (DDGS)).

The methods and compositions of the present disclosure can be adapted to conventional fermentation bioreactors (e.g., batch, fed-batch, cell recycle, and continuous fermentation).

In some embodiments, a yeast (e.g., a genetically modified yeast) as provided herein is cultivated in liquid fermentation media (i.e., a submerged culture) which leads to excretion of the fermented product(s) into the fermentation media. In one embodiment, the fermented end product(s) can be isolated from the fermentation media using any suitable method known in the art.

In some embodiments, formation of the fermented product occurs during an initial, fast growth period of the yeast. In one embodiment, formation of the fermented product occurs during a second period in which the culture is maintained in a slow-growing or non-growing state. In one embodiment, formation of the fermented product occurs during more than one growth period of the yeast. In such embodiments, the amount of fermented product formed per unit of time is generally a function of the metabolic activity of the yeast, the physiological culture conditions (e.g., pH, temperature, medium composition), and the amount of yeast present in the fermentation process.

Ethanol and co-products of interest may be separated and purified by the approaches described in the following paragraphs, taking into account that many methods of separation and purification are known in the art and the following disclosure is not meant to be limiting.

As to general processing of a fermentation broth comprising ethanol and low boiling molecules, various methods may be practiced to remove biomass and/or separate ethanol and low boiling molecules from the culture broth and its components. A sugar-based feedstock stream is converted into ethanol and other co-products of interest in a fermenter as disclosed herein. In an embodiment of the disclosure, ethanol and one or more low-boiling co-products are produced, and these products are obtained both in the vapor phase (offgas) and in the liquid phase (broth). The products in the offgas are recovered in an absorption column or other washing equipment to minimize losses of ethanol and low boiling volatile co-products. This stream with the recovered products from the offgas and the broth can be mixed for further processing. Alternatively, a solid removal step can be performed, comprising centrifugation, decanting, filtering, or a combination thereof, and the operation unit system can be performed depending on the size of the solid particles present in the broth. Optionally, an incondensable gases removal can be adapted comprising of a flash unit, or a distillation unit or an absorption unit or a combination thereof. Following, the mixture can go directly to a distillation column system comprising one or more distillation columns, but depending on the nature of the low boiling molecules, the system can further comprise one or more additional operational units comprising extractive distillation, azeotropic distillation, flash, adsorption and absorption or a combination thereof. At the end of these steps, ethanol and the volatile products are obtained in the specification required for their specific applications.

As to general processing of a fermentation broth comprising ethanol and high boiling molecules, various methods may be practiced to remove biomass and/or separate ethanol and high boiling molecules from the culture broth and its components. The process to isolate the ethanol from the one or more high boiling co-products is conducted by distillation to remove volatiles (especially ethanol) and followed by a process selected from crystallization, solvent extraction, chromatographic separation, adsorption, filtration, salt splitting, sedimentation, acidification, ion exchange, evaporation, or combinations thereof to result in a purified high boiling molecule.

The fermentation products are subjected to a centrifugation unit to sediment cells and insoluble contents. The liquid supernatant phase contains water, ethanol and soluble co-products. In sequence, distillation is applied to separate the volatile products (especially ethanol) as a vapor while the high boiling co-products and salts remain in the liquid aqueous phase. The stream containing the liquid phase is lead to a separation of salts in a process involving one or more of the following possible processes including, but not limited to: crystallization, chromatographic separation, solvent extraction, adsorption, salt splitting, sedimentation, filtration (ultra, nano and/or microfiltration), acidification, ion exchange, or other processes and combinations thereof. The stream containing high boiling products in solution may be concentrated in a simple distillation column or by single-stage evaporation or by multistage evaporation stages, depending on the relative volatility related to other co-products or water. For example, when the high boiling product is dispersed in water, the product will be collected at the bottom of the column, while water will be removed at the top of column. If the high boiling co-product forms azeotrope with water, a set of extraction units or molecular sieves may be required. The recovered product may be finished up in a dryer to decrease humidity and increase stability for further storage.

In another embodiment, the biomass from the carbon source (e.g. unfermented grain residues) is also part of the fermentation broth. The fermentation products are subjected to a distillation process to separate the volatile products (especially ethanol) as a vapor while the high boiling co-products, cell debris, the distillers grains from the carbon source and salts remain in the liquid phase. The products in liquid phase are subjected to a centrifugation unit to sediment cell debris, the insoluble portion of the distiller grains and other insoluble contents. The supernatant phase of the centrifugation process lead to a separation of salts and the soluble portion of the distiller grains from the high boiling molecules in a process involving one or more of the following possible processes including, but not limited to: crystallization, chromatographic separation, solvent extraction, adsorption, salt splitting, sedimentation, filtration (ultra, nano and/or microfiltration), acidification, ion exchange, or other processes and combinations thereof. Streams containing both the soluble and insoluble portions of the distillers grains may be combined and subject to an evaporator unit and/or a dryer to decrease humidity and constitute a dried distillers grains with solubles (DDGS) portion. The stream containing high boiling products in solution may be concentrated in a simple distillation column or by single-stage evaporation or by multistage evaporation stages, depending on the relative volatility related to other co-products or water.

### EXAMPLES

### Example 1: Modification of Ethanol Producer Yeast for Production of 1-Propanol

A yeast is genetically modified to produce 1-propanol from a fermentable carbon source including, for example, glucose.

In an exemplary method, a yeast is genetically engineered by any methods known in the art to comprise: (i) one or more polynucleotides coding for enzymes in a pathway that catalyze a conversion of the fermentable carbon source to succinyl-CoA; (ii) one or more polynucleotides coding for enzymes in a pathway that catalyze a conversion of succinyl-CoA to methylmalonyl-CoA; (iii) one or more polynucleotides coding for enzymes in a pathway that catalyze a conversion of methylmalonyl-CoA to propionyl-CoA; (iv) one or more polynucleotides coding for enzymes in a pathway that catalyze a conversion of propionyl-CoA to propionaldehyde; and (v) one or more polynucleotides coding for enzymes in a pathway that catalyze a conversion of propionaldehyde to 1-propanol.

In another exemplary method a yeast is genetically engineered by any methods known in the art to comprise: (i) one or more polynucleotides coding for enzymes in a pathway that catalyze a conversion of the fermentable carbon source to 1,2-propanediol; (ii) one or more polynucleotides coding for enzymes in a pathway that catalyze a conversion of 1,2-propanediol to propionaldehyde; and (iii) one or more polynucleotides coding for enzymes in a pathway that catalyze a conversion of propionaldehyde to 1-propanol.

In another exemplary method, a yeast is genetically engineered by any methods known in the art to comprise: (i) one or more polynucleotides coding for enzymes in a pathway that catalyze a conversion of the fermentable carbon source to threonine or citramalate; (ii) one or more polynucleotides coding for enzymes in a pathway that catalyze a conversion of threonine or citramalate to 2-ketobutyrate (2-kB); (iii) one or more polynucleotides coding for enzymes in a pathway that catalyze a conversion of 2-kB to propionyl-CoA; (iv) one or more polynucleotides coding for enzymes in a pathway that catalyze a conversion of propionyl-CoA to propionaldehyde; and (v) one or more polynucleotides coding for enzymes in a pathway that catalyze a conversion of propionaldehyde to 1-propanol.

In another exemplary method, a yeast is genetically engineered by any methods known in the art to comprise: (i) one or more polynucleotides coding for enzymes in a pathway that catalyze a conversion of the fermentable carbon source to lactate or β-alanine; (ii) one or more polynucleotides coding for enzymes in a pathway that catalyze a conversion of lactate or β-alanine to acrylyl-CoA; (iii) one or more polynucleotides coding for enzymes in a pathway that catalyze a conversion of acrylyl-CoA to propionyl-CoA; (iv) one or more polynucleotides coding for enzymes in a pathway that catalyze a conversion of propionyl-CoA to propionaldehyde; and (v) one or more polynucleotides coding for enzymes in a pathway that catalyze a conversion of propionaldehyde to 1-propanol.

In another exemplary method, a yeast is genetically engineered by any methods known in the art to comprise: (i) one or more polynucleotides coding for enzymes in a pathway that catalyze a conversion of the fermentable carbon source to β-alanine; (ii) one or more polynucleotides coding for enzymes in a pathway that catalyze a conversion of β-alanine to malonate semialdehyde; (iii) one or more polynucleotides coding for enzymes in a pathway that catalyze a conversion of malonate semialdehyde to 3-hydroxypropionate β-HP); (iv) one or more polynucleotides coding for enzymes in a pathway that catalyze a conversion of 3-HP to acrylyl-CoA; (v) one or more polynucleotides coding for enzymes in a pathway that catalyze a conversion of acrylyl-CoA to propionyl-CoA; (vi) one or more polynucleotides coding for enzymes in a pathway that catalyze a conversion of propionyl-CoA to propionaldehyde; and (vii) one or more polynucleotides coding for enzymes in a pathway that catalyze a conversion of propionaldehyde to 1-propanol.

In another exemplary method, a yeast is genetically engineered by any methods known in the art to comprise: (i) one or more polynucleotides coding for enzymes in a pathway that catalyze a conversion of the fermentable carbon source to oxaloacetate malonate semialdehyde; (ii) one or more polynucleotides coding for enzymes in a pathway that catalyze a conversion of oxaloacetate to malonate semialdehyde; (iii) one or more polynucleotides coding for enzymes in a pathway that catalyze a conversion of malonate semialdehyde to 3-hydroxypropionate β-HP); (iv) one or more polynucleotides coding for enzymes in a pathway that catalyze a conversion of 3-HP to acrylyl-CoA; (v) one or more polynucleotides coding for enzymes in a pathway that catalyze a conversion of acrylyl-CoA to propionyl-CoA; (vi) one or more polynucleotides coding for enzymes in a pathway that catalyze a conversion of propionyl-CoA to propionaldehyde; and (vii) one or more polynucleotides coding for enzymes in a pathway that catalyze a conversion of propionaldehyde to 1-propanol.

Alternatively, a yeast that lacks one or more enzymes (e.g., one or more functional enzymes that are catalytically active) for the conversion of a fermentable carbon source to 1-propanol is genetically modified to comprise one or more polynucleotides coding for enzymes (e.g., functional enzymes including, for example any enzyme disclosed herein) in a pathway that the yeast lacks to catalyze a conversion of the fermentable carbon source to 1-propanol.

### Example 2: Modification of Ethanol Producer Yeast for Production of Acetone, 2-Propanol, Propene, and/or 1-Butanol

In an exemplary method, a yeast is genetically engineered by any methods known in the art to comprise: (i) one or more polynucleotides coding for enzymes in a pathway that catalyze a conversion of the fermentable carbon source to pyruvate or malonate semialdehyde (MSA); (ii) one or more polynucleotides coding for enzymes in a pathway that catalyze a conversion of pyruvate or MSA to acetyl-CoA; (iii) one or more polynucleotides coding for enzymes in a pathway that catalyze a conversion of acetyl-CoA to acetoacetyl-CoA; (iv) one or more polynucleotides coding for enzymes in a pathway that catalyze a conversion of acetoacetyl-CoA to acetoacetate; and (v) one or more polynucleotides coding for enzymes in a pathway that catalyze a conversion of acetoacetate to acetone.

In an exemplary method, a yeast is genetically engineered by any methods known in the art to comprise: (i) one or more polynucleotides coding for enzymes in a pathway that catalyze a conversion of the fermentable carbon source to pyruvate or malonate semialdehyde (MSA); (ii) one or more polynucleotides coding for enzymes in a pathway that catalyze a conversion of pyruvate or MSA to acetyl-CoA; (iii) one or more polynucleotides coding for enzymes in a pathway that catalyze a conversion of acetyl-CoA to malonyl-CoA; (iv) one or more polynucleotides coding for enzymes in a pathway that catalyze a conversion of malonyl-CoA to acetoacetyl-CoA; (v) one or more polynucleotides coding for enzymes in a pathway that catalyze a conversion of acetoacetyl-CoA to acetoacetate; and (vi) one or more polynucleotides coding for enzymes in a pathway that catalyze a conversion of acetoacetate to acetone.

In another exemplary method, a yeast is genetically engineered by any methods known in the art to comprise: (i) one or more polynucleotides coding for enzymes in a pathway that catalyze a conversion of the fermentable carbon source to pyruvate or malonate semialdehyde (MSA); (ii) one or more polynucleotides coding for enzymes in a pathway that catalyze a conversion of pyruvate or MSA to acetyl-CoA; (iii) one or more polynucleotides coding for enzymes in a pathway that catalyze a conversion of acetyl-CoA to acetoacetyl-CoA; (iv) one or more polynucleotides coding for enzymes in a pathway that catalyze a conversion of acetoacetyl-CoA to hydroxymethylglutaryl-CoA (HMG-CoA); (v) one or more polynucleotides coding for enzymes in a pathway that catalyze a conversion of HMG-CoA to acetoacetate; and (vi) one or more polynucleotides coding for enzymes in a pathway that catalyze a conversion of acetoacetate to acetone.

In an exemplary method, a yeast is genetically engineered by any methods known in the art to comprise: (i) one or more polynucleotides coding for enzymes in a pathway that catalyze a conversion of the fermentable carbon source to pyruvate or malonate semialdehyde (MSA); (ii) one or more polynucleotides coding for enzymes in a pathway that catalyze a conversion of pyruvate or MSA to acetyl-CoA; (iii) one or more polynucleotides coding for enzymes in a pathway that catalyze a conversion of acetyl-CoA to malonyl-CoA; (iv) one or more polynucleotides coding for enzymes in a pathway that catalyze a conversion of malonyl-CoA to acetoacetyl-CoA; (v) one or more polynucleotides coding for enzymes in a pathway that catalyze a conversion of acetoacetyl-CoA to hydroxymethylglutaryl-CoA (HMG-CoA); (vi) one or more polynucleotides coding for enzymes in a pathway that catalyze a conversion of HMG-CoA to acetoacetate; and (vii) one or more polynucleotides coding for enzymes in a pathway that catalyze a conversion of acetoacetate to acetone.

In another exemplary method, a yeast is genetically engineered by any methods known in the art to comprise: (i) one or more polynucleotides coding for enzymes in a pathway that catalyze a conversion of the fermentable carbon source to acetone; and (ii) one or more polynucleotides coding for enzymes in a pathway that catalyze a conversion of acetone to isopropanol (2-propanol).

In another exemplary method, a yeast is genetically engineered by any methods known in the art to comprise: (i) one or more polynucleotides coding for enzymes in a pathway that catalyze a conversion of the fermentable carbon source to butyrate; (ii) one or more polynucleotides coding for enzymes in a pathway that catalyze a conversion of butyrate to propane; and (iii) one or more polynucleotides coding for enzymes in a pathway that catalyze a conversion of propane to 2-propanol.

In another exemplary method, a yeast is genetically engineered by any methods known in the art to comprise: (i) one or more polynucleotides coding for enzymes in a pathway that catalyze a conversion of the fermentable carbon source to 2-propanol; and (ii) one or more polynucleotides coding for enzymes in a pathway that catalyze a conversion of 2-propanol to propene.

In another exemplary method, a yeast is genetically engineered by any methods known in the art to comprise: (i) one or more polynucleotides coding for enzymes in a pathway that catalyze a conversion of the fermentable carbon source to butyrate or butyryl-CoA; (ii) one or more polynucleotides coding for enzymes in a pathway that catalyze a conversion of butyrate or butyryl-CoA to butanal; and (iii) one or more polynucleotides coding for enzymes in a pathway that catalyze a conversion of butanal to 1-butanol.

Alternatively, a yeast that lacks one or more enzymes (e.g., one or more functional enzymes that are catalytically active) for the conversion of a fermentable carbon source to acetone, 2-propanol, propene, and/or 1-butanol is genetically modified to comprise one or more polynucleotides coding for enzymes (e.g., functional enzymes including, for example any enzyme disclosed herein) in a pathway that the yeast lacks to catalyze a conversion of the fermentable carbon source to acetone, 2-propanol, propene, and/or 1-butanol.

### Example 3: Fermentation of Glucose by Genetically Modified Ethanol Producer Yeast to Produce 1-Propanol, Acetone, 2-Propanol, Propene, and/or 1-Butanol

A genetically modified yeast, as produced in Example 1 or Example 2 above, is used to ferment a carbon source to produce 1-propanol, acetone, 2-propanol, propene, and/or 1-butanol.

In an exemplary method, a previously-sterilized culture medium comprising a fermentable carbon source (e.g., 9 g/L glucose, 1 g/L KH₂PO₄, 2 g/L (NH₄)₂HPO₄, 5 mg/L FeSO₄.7H₂O, 10 mg/L MgSO₄.7H₂O, 2.5 mg/L MnSO₄.H₂O, 10 mg/L CaCl₂.6H₂O, 10 mg/L CoCl₂.6H₂O, and 10 g/L yeast extract) is charged in a bioreactor.

During fermentation, anaerobic conditions are maintained by, for example, sparging nitrogen through the culture medium. A suitable temperature for fermentation (e.g., about 30° C.) is maintained using any method known in the art. A near physiological pH (e.g., about 6.5) is maintained by, for example, automatic addition of sodium hydroxide. The bioreactor is agitated at, for example, about 50 rpm. Fermentation is allowed to run to completion.

### Example 4: Fermentation of Glucose by Genetically Modified Ethanol Producer Yeast to Produce Ethanol and Low Boiling Co-Products

A genetically modified yeast, as produced in Example 1 or Example 2 above, is used to ferment a carbon source to produce ethanol and one or more low boiling co-products such as 1-propanol, 2-propanol, acetone, methyl ethyl ketone, ethyl acetate, isopropyl acetate, ethane, and propene.

In an exemplary method, a previously-sterilized culture medium comprising a fermentable carbon source (e.g., 9 g/L glucose, 1 g/L KH₂PO₄, 2 g/L (NH₄)₂HPO₄, 5 mg/L FeSO₄.7H₂O, 10 mg/L MgSO₄.7H₂O, 2.5 mg/L MnSO₄.H₂O, 10 mg/L CaCl₂.6H₂O, 10 mg/L CoCl₂.6H₂O, and 10 g/L yeast extract) is charged in a bioreactor.

During fermentation, anaerobic conditions, if used, are maintained by, for example, sparging nitrogen through the culture medium. A suitable temperature for fermentation (e.g., about 30° C.) is maintained using any method known in the art. A near physiological pH (e.g., about 6.5) is maintained by, for example, automatic addition of sodium hydroxide. The bioreactor is agitated at, for example, about 50 rpm. Fermentation is allowed to run to completion.

### Example 5: Fermentation of Glucose by Genetically Modified Ethanol Producer Yeast to Produce Ethanol and High Boiling Co-Products

A genetically modified yeast, as produced in Example 1 or Example 2 above, is used to ferment a carbon source to produce ethanol and one or more high boiling co-products such as monoethylene glycol, n-butanol, 3-hydroxypropionic acid, adipic acid, diethanolamine, and 1,3-propanediol.

In an exemplary method, a previously-sterilized culture medium comprising a fermentable carbon source (e.g., 9 g/L glucose, 1 g/L KH₂PO₄, 2 g/L (NH₄)₂HPO₄, 5 mg/L FeSO₄.7H₂O, 10 mg/L MgSO₄.7H₂O, 2.5 mg/L MnSO₄.H₂O, 10 mg/L CaCl₂.6H₂O, 10 mg/L CoCl₂.6H₂O, and 10 g/L yeast extract) is charged in a bioreactor.

During fermentation, anaerobic conditions, if used, are maintained by, for example, sparging nitrogen through the culture medium. A suitable temperature for fermentation (e.g., about 30° C.) is maintained using any method known in the art. A near physiological pH (e.g., about 6.5) is maintained by, for example, automatic addition of sodium hydroxide. The bioreactor is agitated at, for example, about 50 rpm. Fermentation is allowed to run to completion.

### Example 6: Effect of High Concentrations of C3 and C4 Alcohols on Yeast

An alcohol tolerance experiment was conducted to understand the negative effects of n-propanol (i.e., 1-propanol), 2-propanol, and 2-butanol compared to ethanol in Saccharomyces cerevisiae. Ethanol, which is a natural product (or native product) produced during sugar-ethanol fermentation is generally well-tolerated by yeast such as S. cerevisiae. However, existing approaches to produce non-natural chemicals such as C3, C4, or C5 alcohols, ketones, organic acids, or other non-natural products (e.g., alcohols other than ethanol) by using genetically modified yeast are usually impacted negatively by the higher toxicity compared to ethanol of such non-natural chemicals or alcohols (e.g., n-propanol and 2-propanol) to the yeast cell-growth and/or performance.

Several concentrations of n-propanol, 2-propanol, 2-butanol and ethanol was tested in yeast cultures. The experiment was conducted using the industrial ethanol-producing yeast strain PE-2 in a 250 mL shaken flask with 50 mL of YNB medium having 40 g/L glucose at 32° C. The culture was conducted during 8-9 hours with an initial OD₆₀₀ₙₘ=12 (OD=optical density). Samples were taken in adequate intervals and analyzed by HPLC to measure glucose, ethanol, n-propanol, 2-propanol and 2-butanol. The experiment was performed according to the parameters in Table 13, in duplicate. The conditions at which no sugar consumption was observed were considered a lethal concentration and were excluded from the analysis.

TABLE 13
Experimental Design - Yeast tolerance to C2, C3 and C4 alcohols.

| Condition | Alcohol | | Sugar |
|---|---|---|---|
| No. | Concentration | Flask Label | Consumption |
| 1 | Control (+) No | Control (+) | Yes |
| | alcohol added | | |
| 2 | Ethanol 20 g/L | ETOH 20 g/L | Yes |
| 3 | Ethanol 40 g/L | ETOH 40 g/L | Yes |
| 4 | Ethanol 60 g/L | ETOH 60 g/L | Yes |
| 5 | Ethanol 80 g/L | ETOH 80 g/L | Yes |
| 6 | Ethanol 120 g/L | ETOH 120 g/L | Yes |
| 7 | n-Propanol 20 g/L | PROP 20 g/L | Yes |
| 8 | n-Propanol 40 g/L | PROP 40 g/L | Yes |
| 9 | n-Propanol 60 g/L | PROP 60 g/L | Yes |
| 10 | n-Propanol 80 g/L | PROP 80 g/L | No |
| 11 | 2-Propanol 20 g/L | 2-Prop 20 g/L | Yes |
| 12 | 2-Propanol 40 g/L | 2-Prop 40 g/L | Yes |
| 13 | 2-Propanol 60 g/L | 2-Prop 60 g/L | Yes |
| 14 | 2-Propanol 80 g/L | 2-Prop 80 g/L | Yes |
| 17 | 2-Butanol 20 g/L | 2-But 20 g/L | Yes |
| 18 | 2-Butanol 40 g/L | 2-But 40 g/L | Yes |
| 19 | 2-Butanol 60 g/L | 2-But 60 g/L | No |
| 20 | 2-Butanol 80 g/L | 2-But 80 g/L | No |

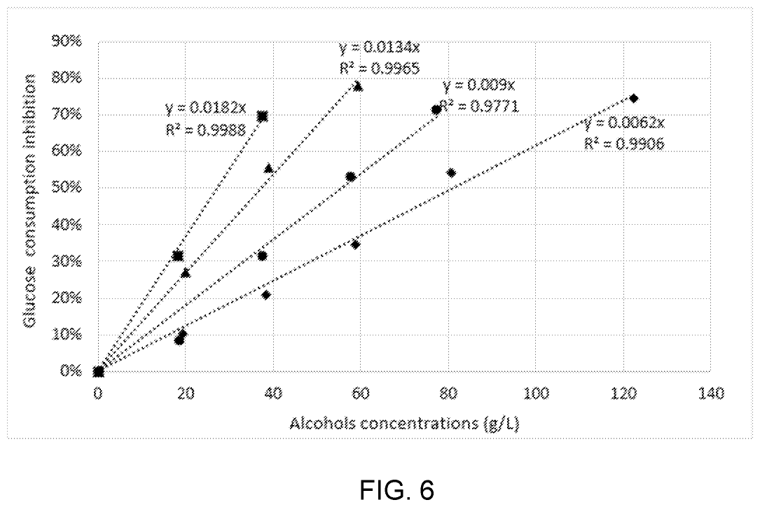

The percentage of glucose consumption inhibition was assessed for various concentrations of alcohols. Samples were tested two hours after inoculation. At this point, glucose had not been totally consumed. The linear regression curve is shown in FIG. 6 and the results are provided in Table 14.

TABLE 14
Sugar consumption inhibition dependence on alcohol concentrations.

| | | | Sugar consumption inhibition | |
|---|---|---|---|---|
| | | | per unit of alcohol regarding | |
| | | Coefficient of | the Control (+) condition | Toxicity |
| | | determination | (no alcohol added) | related to |
| Alcohol | Slope | R² | (% per g · L⁻¹ of alcohol) | ethanol |
| Ethanol | 0.0062 | 0.9906 | 0.62% | — |
| 2-Propanol | 0.009 | 0.9771 | 0.90% | 1.45 |
| n-Propanol | 0.0134 | 0.9965 | 1.34% | 2.16 |
| 2-Butanol | 0.0182 | 0.9988 | 1.82% | 2.94 |

As observed in FIG. 6, n-propanol, 2-propanol and 2-butanol, which are non-natural in S. cerevisiae, negatively affect S. cerevisiae and the effect is greater at high concentration. As shown in Table 14, 2-butanol showed 2.94 times more inhibition than ethanol. On the other hand, 2-propanol showed 1.45 times more inhibition than ethanol. N-propanol showed an intermediate effect between 2-propanol and 2-butanol, with 2.16 times more inhibition than ethanol. These results demonstrate how non-natural products like n-propanol, 2-propanol and 2-butanol can promote a negative effect on yeast such as Saccharomyces cerevisiae, compromising sugar consumption profiles and therefore aspects of ethanol fermentation performance such as productivity.

### Example 7: Simulation of an Industrial Ethanol Yeast-Fermentation Performance Wherein 1-Propanol and 2-Propanol are Co-Produced at Non-Toxic Concentrations with Ethanol as a Major Component

A laboratory simulation was done to study the effects on a yeast sugar-ethanol fermentation wherein a co-product, or a non-natural product in yeast, is produced along with ethanol. Two conditions were tested: i) condition 1, wherein the industrial ethanol-producing yeast produces ethanol from sugar added in the culture media and at the same time additional ethanol was exogenously added aiming to reach the expected final ethanol titer; and ii) condition 2, wherein the same industrial ethanol-producing yeast produces ethanol from sugar added in the culture media and a concentrated solution of n-propanol and 2-propanol (50/50 wt. %) was exogenously added in the culture media in order to reach the same final titer concentration of products than condition 1. The experiment was performed using a 1 L bioreactor with 0.7 L as a final volume. The pH was controlled at 4.5 by adding NaOH 25% w/w, 32° C. temperature and 300 rpm stirring. The industrial ethanol-producing yeast strain used was PE-2, with an initial pitch of 0.7 g/L DWC and the culture medium was YNB without amino acids. The final sugar concentration was 224 g/L glucose. The experiment was performed under aseptic conditions.

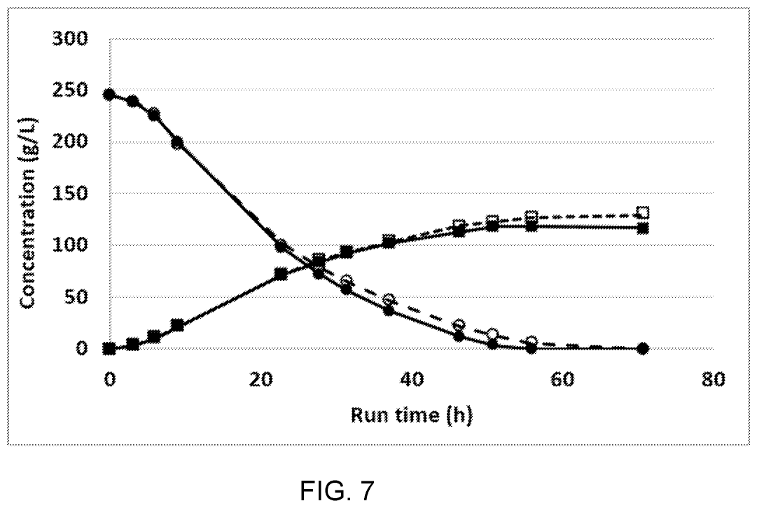

The bioreactor was first filled with 650 ml of YNB medium plus sugar, and after pH and temperature stabilization, a suspension of 50 mL with the yeast inoculum was added into the bioreactor. Then, 130 mL of the concentrated ethanol solution of 160 g/L was added for Condition 1, and 130 mL of the concentrated n-propanol and 2-propanol solution of 177 g/L was added for Condition 2. Each solution followed the profile: 10 hours since inoculation, 0.2 mL/min; from 11 hours to 15 hours, 0.4 mL/min; from 16 hours to 40 hours, 0.6 mL/min; and from 41 hours to 46 hours, 0.2 mL/min. This profile was added to simulate an ethanol production profile of PE-2 yeast. The fermentation was ended with 70 hours of fermentation run. Samples were taken in adequate intervals to measure ethanol, 1-propanol, 2-propanol and glucose. Results are presented in Table 15 and FIG. 7.

TABLE 15
Ethanol-yeast fermentation with added alcohols.

| | | | | | | | | Fraction | | |
|---|---|---|---|---|---|---|---|---|---|---|
| | | | | | | Total | Final | of | | |
| | Sugar | Ethanol | Ethanol | 1- | 2- | alcohol | total | Alcohol | Ethanol | Volumetric |
| | added | produced | added | propanol | propanol | added | Alcohols | added | yield | productivity |
| Condition | (g) | (g) | (g) | added (g) | added (g) | (g) | (g) | (%) | (g/g) | (g/Lh) |
| 1 | 179.6 | 80.9 | 15.9 | 0.0 | 0.0 | 15.9 | 96.8 | 16.4 | 0.5 | 1.4 |
| 2 | 180.1 | 81.1 | 0.0 | 8.2 | 7.9 | 16.1 | 97.2 | 16.5 | 0.5 | 1.5 |

As shown in Table 15, the yeast fermentation parameters of ethanol yield and volumetric productivity were similar for both conditions tested. In other words, the yeast ethanol fermentation profile for the yeast culture exposed only to ethanol (Condition 1) was similar to that exposed to a mixture of n-propanol and 2-propanol (Condition 2). Minimal or no impact on ethanol yield and volumetric productivity was observed under Condition 2 (wherein 16.5% of the total final alcohols in the fermentation were C3 alcohols, a combination of n-propanol and 2-propanol) compared to Condition 1. In addition, as shown in FIG. 7, sugar consumption and ethanol production are similar for both tested conditions. These results demonstrate that the tested concentrations of n-propanol and 2-propanol avoid compromising yeast fermentation performance, as assessed by ethanol fermentation yield and volumetric productivity, during an ethanol fermentation.

### Example 8: Recombinant Ethanol-Producing Yeast Co-Producing 3-Hydroxypropionic Acid with Ethanol as a Major Component During Ethanol Fermentation from Glucose

An ethanol-producing S. cerevisiae yeast strain was genetically modified to co-produce 3-hydroxypropionic acid with ethanol as a major component through a carbon flow redirection from glucose as a carbon source. Saccharomyces cerevisiae is not naturally capable of producing 3-hydroxypropionic acid from glucose. Therefore, a 3-hydroxypropionic acid producing metabolic pathway and target enzymes were heterologously expressed into a Saccharomyces cerevisiae yeast (W303 strain). Additionally, the yeast strain was modified to downregulate the natural ethanol-producing metabolic pathway in the pyruvate node by the deletion of the wild-type pyruvate kinase (PYK1) and expression of a PYK1 enzyme downregulated using weaker promoters (pNUP57 and pMET25ΔF) to decrease PYK1 enzyme half-life and thereby reduce the carbon flow from PEP towards pyruvate and better control the amount of ethanol naturally produced.

As shown in Table 16, recombinant yeast strains YS_001 and YS_002 had 3-hydroxypropionic acid pathway producing genes integrated into the genome, including AAT2 from S. cerevisiae (AAT2.Sc), PAND from T. castaneum (PAND.Tca), PYD4 from L. kluyveri (PYD4.Lk), and YDFG from E. coli (YDFG.Ec). In addition, these strains have PEP.CK from E. coli (PEPCK.Ec) over-expressed to redirect carbon flow from PEP to oxaloacetate (OAA). All the 3-hydroxypropionic acid biosynthetic pathway genes were codon-optimized to be optimally expressed in yeast, under the control of promoters of varied strengths and also varying the number of gene copies.

An ethanol fermentation test was performed in the presence of 25 mL of YPD media with 80 g/L glucose in 125 mL fermentation flask. Stirring was at 135 rpm on 50 mm shaking diameter incubators. 3-hydroxypropionic acid, ethanol, glycerol and glucose were measured after 48 hours fermentation using standard analytical methods and equipment and the results are shown in Table 16.

TABLE 16
Co-production of 3-hydroxypropionic acid with ethanol as a major
component during anaerobic ethanol fermentation from glucose.

| Yeast | | | | Glucose | 3-HP | Ethanol | Glycerol |
|---|---|---|---|---|---|---|---|
| Strain | Genotype | Phenotype | OD600 nm | (g/L) | (g/L) | (g/L) | (g/L) |
| YS_001 | jlpl1::[TRP1.Kl-loxP- | MET- | 61 | 0 | 4.7 | 29 | 3 |
| | pMET25-PYK1], | | | | | | |
| | met14::[HIS3.Sba-RS- | | | | | | |
| | PAND.Tca-PYD4.Lk- | | | | | | |
| | YDFG.Ec], | | | | | | |
| | pyk1::[LEU2.Kl-loxP- | | | | | | |
| | PEPCK.Ec-AAT2.Sc- | | | | | | |
| | PEPCK.Ec], | | | | | | |
| | ura3::[PAND.Tca- | | | | | | |
| | YDFG.Ec-URA3]x10 | | | | | | |
| YS_002 | jlp1::[pNUP57-PYK1], | MET- | 23 | 50 | 7.5 | 5 | 5 |
| | pyk1::[PEPCK.Ec- | | | | | | |
| | AAT2.Sc-PEPCK.Ec], | | | | | | |
| | met14::[PAND.Tca- | | | | | | |
| | PYD4.Lk-YDFG.Ec], | | | | | | |
| | ura3::[PAND.Tca- | | | | | | |
| | YDFG.Ec]x9 | | | | | | |

Recombinant yeast strain YS_001 used a slightly stronger promoter (pMET25ΔF) for PYK1 expression allowing an adequate control of sugar ratio from glucose towards either ethanol as a major component or 3-hydroxypropionic acid as a by-product at non-toxic amounts, leading to a desired sugar-ethanol fermentation profile. YS_001 was capable of consuming all glucose fed showing a very good cell growth reaching a final OD600 of 61 despite of the genetic modifications to redirect carbon flow from glucose to either ethanol or 3-hydroxypropionic acid and also to introduce heterologous genes for production of non-natural 3-hydroxypropionic acid with ethanol. YS_001 recombinant yeast strain was able to co-produce 4.7 g/L of 3-hydroxypropionic acid with ethanol at high concentration of 29 g/L. In summary, the results in Table 16 show that 3-hydroxypropionic acid was co-produced with ethanol as a major component during a sugar-ethanol fermentation wherein the ratio of products was controlled to retain ethanol performance while producing 3-hydroxypropionic acid at a low and non-toxic concentration.

Although the results presented herein were demonstrated using the recombinant yeast strain W303, other Saccharomyces cerevisiae yeast strains including industrial yeasts such as PE-2, CAT-1, BG-1 and Ethanol Red yeast strains, which are widely used in industrial sugarcane-ethanol and corn-ethanol fermentation processes, can also be used.

### Example 9: Recombinant Ethanol-Producing Yeast Co-Producing 1-Propanol with Ethanol as a Major Component During Ethanol Fermentation from Glucose

An ethanol-producing S. cerevisiae yeast strain was genetically modified to co-produce 1-propanol with ethanol as a major component through a carbon flow redirection from glucose as a carbon source. Saccharomyces cerevisiae is naturally capable of producing only residual amounts of 1-propanol via the Ehrlich pathway involved in the branched-chain amino acids metabolism. A 1-propanol-producing biosynthetic metabolic pathway and target enzymes were heterologously expressed in the W303 yeast strain. Additionally, the yeast strain was modified to downregulate the natural ethanol-producing metabolic pathway in the pyruvate node by the deletion of the wild-type pyruvate kinase (PYK1) and expression of a PYK1 enzyme downregulated using a weak promoter such as pNUP57 to decrease PYK1 enzyme half-life and thereby reduce the carbon flow from PEP towards pyruvate and better control the amount of ethanol naturally produced.

As shown in Table 17, recombinant yeast strains YS_003 and YS_004 had 1-propanol pathway producing genes integrated into the genome in varied copies, including AAT2 from S. cerevisiae (AAT2.Sc), PAND from T. castaneum (PAND.Tca), PYD4 from L. kluyveri (PYD4.Lk), YDFG from E. coli (YDFG.Ec), HPD1 from C. albicans (HPD1.Ca), PCT from C. propionicum (PCT.Cp), HPCD and ACR from R. pomeroyi (HPCD.Rp and ACR.Rp), and PDUP from S. enterica (PDUP.Sen). All the 1-propanol biosynthetic pathway genes were codon-optimized to be optimally expressed in yeast and the constructed recombinant yeast strains had PEP.CK from E. coli (PEPCK.Ec) over-expressed to redirect carbon flow from PEP to oxaloacetate (OAA). YS_003 and YS_004 had different 3-hydroxypropionic acid dehydrogenase candidates (3HPDH) responsible for the conversion of MSA into 3-hydroxypropionic acid. YS_003 had a NADPH-dependent 3HPDH enzyme (YDFG.Ec), while YS_004 had a NADH-dependent 3HPDH enzyme (HPD1.Cal) over-expressed.

An ethanol fermentation test was performed in the presence of 25 mL of rich media with 40 g/L glucose in 125 mL fermentation flask plugged with a silicon cap pierced with two pipettes tips of 1 mL with filter. Another 40 g/L of glucose was added after 24 hours of growth. Stirring was at 180 rpm on 50 mm shaking diameter incubators. 1-Propanol, ethanol, glycerol and glucose were measured after 48 hours fermentation using standard analytical methods and equipment (GC/MS-MS) and the results are shown in Table 17.

TABLE 17
Co-production of 1-propanol with ethanol as a major
component during ethanol fermentation from glucose.

| Yeast | | | Glucose | 1-propanol | Ethanol | Glycerol |
|---|---|---|---|---|---|---|
| Strain | Genotype | OD600 nm | (g/L) | (g/L) | (g/L) | (g/L) |
| YS_003 | jlp1::[MET14.Sba-PDUP.Sen- | 43 | 0 | 0.71 | 30 | <2 |
| | PDUP.Sen-PDUP.Sen-ACR.Rp- | | | | | |
| | ACR.Rp-HPCD.Rp-HPCD.Rp- | | | | | |
| | PCT.Cp-PCT.Cp-PCT.Cp- | | | | | |
| | PCT.Cp- pNUP57-PYK1], | | | | | |
| | met14::[HIS3.Sba-PAND.Tca- | | | | | |
| | PYD4.Lk-YDFG.Ec], pyk1::[loxP- | | | | | |
| | PEPCK.Ec-AAT2.Sc-PEPCK.Ec], | | | | | |
| | ura3::[PAND.Tea-YDF1]x5 | | | | | |
| YS_004 | Jpl1::[MET14.Sba-PDUP.Sen- | 59 | 0 | 1.13 | 28 | <2.5 |
| | PDUP.Sen- PDUP.Sen-ACR.Rp- | | | | | |
| | HPCD.Rp- HPCD.Rp -PCT.Cp- | | | | | |
| | PCT.Cp-PCT.Cp-PCT.Cp- | | | | | |
| | pNUP57-PYK1], | | | | | |
| | met14::[HIS3.Sba-RS-PAND.Tca- | | | | | |
| | PYD4.Lk-YDFG.Ec], | | | | | |
| | pyk1::[LEU2.K1-PEPCK.Ec], trp1, | | | | | |
| | ura3::[PAND.Tca-HPD1.Cal]x7 | | | | | |

YS_003 and YS_004 recombinant yeast strains were able to consume all glucose fed showing relatively good cell growth, reaching a final OD600 of 43 and 59 respectively, despite the genetic modifications to produce 1-propanol and redirect carbon flow from glucose. YS_003 and YS_004 recombinant yeast strains were able to produce 0.71 g/L and 1.13 g/L of 1-propanol respectively, during the ethanol fermentation, while producing ethanol as a major component at high titers of 28-30 g/L according to the amount of glucose fed, 80 g/L. Without wishing to be bound by theory, it is believed that the increased 1-propanol production for YS_004 is due to the higher number of copies of the aspartate decarboxylase and the over-expression of the NADH-dependent 3-hydroxypropionic acid dehydrogenase enzyme.

Although the results presented herein were demonstrated using the recombinant yeast strain W303, other Saccharomyces cerevisiae yeast strains including industrial yeasts such as PE-2, CAT-1, BG-1 and Ethanol Red yeast strains, which are widely used in industrial sugarcane-ethanol and corn-ethanol fermentation processes, can also be used.

### Example 10: Recombinant Ethanol-Producing Yeast Co-Producing Acetone with Ethanol as a Major Component During Ethanol Fermentation from Glucose

An ethanol-producing S. cerevisiae yeast strain was genetically modified to co-produce acetone with ethanol as a major component through a carbon flow redirection from glucose as a carbon source. An acetone-producing metabolic pathway and target enzymes were heterologously expressed into the W303 yeast strain. As shown in Table 18, recombinant yeast strains YS_006 and YS_007 were derived from YS_005 and had acetone pathway producing genes integrated into the genome, including AAT2 from S. cerevisiae (AAT2.Sc), PAND from T. castaneum (PAND.Tca), PYD4 from L. kluyveri (PYD4.Lk), MSD from P. aeruginosa and from C. albicans (MSD.PA or MSD.Cal), ERG10 from S. cerevisiae (ERG10.Sc), ATOAD from E. coli (ATOA.EC and ATOD.Ec), ADC from C. acetobutylicum (ADC.Ca), PTA from C. glutamicum (PTA.Cg), and ACK from E. coli (ACK.Ec). All the acetone biosynthetic pathway genes were codon-optimized to be optimally expressed in yeast and the constructed recombinant yeast strains had PEP.CK from E. coli (PEPCK.Ec) over-expressed to redirect carbon flow from PEP to oxaloacetate (OAA).

An ethanol fermentation test was performed in the presence of 25 mL of rich media with 80 g/L glucose in 125 mL fermentation flask with a silicon cap, where two pipette tips with filter were inserted. Stirring was maintained at 135 rpm on 50 mm shaking diameter incubators. Acetone, ethanol and glucose were measured after 48 hours fermentation using standard analytical methods and equipment (GC/MS-MS headspace). As the parent strain YS_005 lacked heterologous genes and related enzymes to the biosynthesis of acetone, the YS_005 strain was used as negative control at the fermentation assays.

TABLE 18
Co-production of acetone with ethanol as a major component
during ethanol fermentation from glucose.

| Yeast | | | Glucose | Acetone | Ethanol |
|---|---|---|---|---|---|
| Strain | Genotype | OD600 nm | (g/L) | (g/L) | (g/L) |
| YS_005 | met14::[HIS3.Sba-RS-PAND.Tca-PYD4.Lk- | 51 | 0.0 | 0.0 | 39 |
| | PEPCK.Ec-AAT2.Sc] | | | | |
| YS_006 | met14::[HIS3.Sba-RS-PAND.Tca-PYD4.Lk- | 69 | 0.0 | 0.7 | 34 |
| | PEPCK.Ec-AAT2.Sc], Jpl1::[LEU2.Sba-RS- | | | | |
| | MSD.Pa-MSD.Cal-ERG10-ATOA-0.Ec-ATOD- | | | | |
| | 0.Ec-ADC.Ca-PTA.Cg-ACKA.Ec], leu2 | | | | |
| YS_007 | met14::[HIS3.Sba-RS-PAND.Tca-PYD4.Lk- | 75 | 0.0 | 1.0 | 35 |
| | PEPCK.Ec-AAT2.Sc], Jpl1::[LEU2.Sba-RS- | | | | |
| | MSD.Pa-MSD.Cal-ERG10.sc-ATOA.Ec- | | | | |
| | ATOD.Ec-ADC.Ca-PTA.Cg-ACKA.Ec], leu2, | | | | |
| | ura3::[PAND.Tca-MSD.Pa-URA3]x2 | | | | |

YS_006 and YS_007 recombinant yeast strains were able to consume all glucose fed and showed good cell growth reaching a final OD600 of >65 despite the genetic modifications to redirect carbon flow from glucose to ethanol and also to introduce heterologous genes for production of acetone with ethanol. YS_006 and YS_007 recombinant yeast strains were able to produce 0.7 g/L and 1.0 g/L of acetone, respectively, while also maintaining ethanol performance by reaching a high titer of around 35 g/L ethanol, which is very close to the amount produced by the YS_005 strain that is unable to biosynthesize acetone. The results also demonstrated an expected increased production of acetone in the YS_007 strain that comprises additional copies of PAND.Tca and MSD.Pa, which, while not wishing to be bound be theory, is believed to boost the conversion of β-alanine to MSA and MSA to acetyl-CoA, the main acetone precursor.

Although the results presented herein were demonstrated using the recombinant yeast strain W303, other Saccharomyces cerevisiae yeast strains including industrial yeasts such as PE-2, CAT-1, BG-1 and Ethanol Red yeast strains, which are widely used in industrial sugarcane-ethanol and corn-ethanol fermentation processes, can also be used.

### Example 11: Recombinant Ethanol-Producing Yeast Co-Producing 2-Propanol with Ethanol as a Major Component During Ethanol Fermentation from Glucose

An ethanol-producing S. cerevisiae yeast strain was genetically modified to co-produce 2-propanol with ethanol as a major component through a carbon flow redirection from glucose as a carbon source. A 2-propanol-producing metabolic pathway and target enzymes were heterologously expressed into W303 yeast strain. As shown in Table 19, recombinant yeast strain YS_008 had 2-propanol pathway producing genes integrated into the genome, including AAT2 from S. cerevisiae (AAT2.Sc), PAND from T. castaneum (PAND.Tca), PYD4 from L. kluyveri (PYD4.Lk), MSD from P. aeruginosa and from C. albicans (MSD.PA or MSD.Cal), ERG10 from S. cerevisiae (ERG10.Sc), ATOAD from E. coli (ATOA.EC and ATOD.Ec), ADC from P. polymyxa (ADC.Pp), PTA from C. glutamicum (PTA.Cg), ACK from E. coli (ACK.Ec), and IPDH1 from C. beijerinckii (IPDH1.Cbe). All the 2-propanol biosynthetic pathway genes were codon-optimized to be optimally expressed in yeast and the constructed recombinant yeast strains had PEP. CK from E. coli (PEPCK.Ec) over-expressed to also redirect carbon flow from PEP to oxaloacetate (OAA).

An ethanol fermentation test was performed in the presence of 25 mL of rich media with 80 g/L glucose in 125 mL fermentation flask with a silicon cap, where two pipette tips with filter were inserted. Stirring was maintained at 135 rpm on 50 mm shaking diameter incubators. 2-propanol, ethanol and glucose were measured after 48 hours fermentation using standard analytical methods and equipment (GC/MS-MS).

TABLE 19
Co-production of 2-propanol with ethanol as a major
component during ethanol fermentation from glucose.

| Yeast | | | Acetone | 2-propanol | Ethanol |
|---|---|---|---|---|---|
| Strain | Genotype | OD600 nm | (g/L) | (g/L) | (g/L) |
| YS_008 | met14::[HIS3.Sba-PAND.Tca-PYD4.Lk- | 100 | <0.05 | 1.42 | 39 |
| | PEPCK.Ec-AAT2.Sc], jlp1::[LEU2.Sba- | | | | |
| | MSD.Pa-MSD.Cal-ERG10.Sc-ATOA.Ec- | | | | |
| | ATOD.Ec-ADC.Pp-PTA.Cg-ACKA.Ec], | | | | |
| | ura3::[PAND.Tca-MSD.Pa]x5, | | | | |
| | leu2::[MET14.Sba-RS-IPDH1.Cbe] | | | | |

YS_008 recombinant yeast was able to reach a final OD600 of 100 despite the genetic modifications to redirect carbon flow from glucose to ethanol and also to introduce heterologous genes for production of 2-propanol with ethanol. YS_008 recombinant yeast was able to produce 1.42 g/L of 2-propanol and 39 g/L of ethanol, maintaining a good ethanol performance based on the g/L glucose fed.

Although the results presented herein were demonstrated using the recombinant yeast strain W303, other Saccharomyces cerevisiae yeast strains including industrial yeasts such as PE-2, CAT-1, BG-1 and Ethanol Red yeast strains, which are widely used in industrial sugarcane-ethanol and corn-ethanol fermentation processes, can also be used.

### Example 12: Recombinant Ethanol-Producing Yeast Co-Producing Both 1-Propanol and 2-Propanol with Ethanol as a Major Component During Ethanol Fermentation from Glucose

An ethanol-producing S. cerevisiae yeast strain was genetically modified to co-produce 1-propanol and 2-propanol with ethanol as a major component through a carbon flow redirection from glucose as a carbon source. 1-propanol and 2-propanol producing metabolic pathways and target enzymes were heterologously expressed into the W303 yeast strain. Additionally, the yeast strain was modified to downregulate the natural ethanol-producing metabolic pathway in the pyruvate node by the deletion of the wild-type pyruvate kinase (PYK1) and expression of a PYK1 enzyme downregulated using a weak promoter such as pNUP57 to decrease PYK1 enzyme half-life and thereby reduce the carbon flow from PEP towards pyruvate and better control the amount of ethanol naturally produced.

As shown in Table 20, recombinant yeast strain YS_009 had 1-propanol pathway and 2-propanol pathway producing genes integrated into the genome, including AAT2 from S. cerevisiae (AAT2.Sc), PAND from T. castaneum (PAND.Tca), PYD4 from L. kluyveri (PYD4.Lk), YDFG from E. coli (YDFG.Ec), YDF1 from S. cerevisiae (YDF1.Sc), PCT from C. propionicum (PCT.Cp), HPCD and ACR from R. pomeroyi (HPCD.Rp and ACR.Rp), PDUP from S. enterica (PDUP.Sen), MSD from P. aeruginosa and from C. albicans (MSD.Pa or MSS.Ca), ERG10 from S. cerevisiae (ERG10.Sc), ATOAD from E. coli (ATOA.Ec and ATOD.Ec), ADC from P. polymyxa (ADC.Pp), PTA from C. glutamicum (PTA.Cg), ACK from E. coli (ACK.Ec) and IPDH1 from C. beijerinckii (IPDH1.Cbe). In addition, YS_009 had PEP.CK from E. coli (PEPCK.Ec) over-expressed to redirect carbon flow from PEP to oxaloacetate (OAA). All the 1-propanol and 2-propanol biosynthetic pathway genes were codon-optimized to be optimally expressed in yeast, under the control of promoters of varied strengths and also varying the number of gene copies.

TABLE 20
Co-production of 1-propanol and 2-propanol with ethanol as
a major component during ethanol fermentation from glucose.

| Yeast | |
|---|---|
| Strain | Genotype |
| YS_009 | jlp1::[TRP1.K1-PYK1], jlp1::[LEU2.Sba-MSD.Pa-MSD.Cal-ERG10.Sc- |
| | ATOA.Ec-ATOD.Ec-ADC.Pp-PTA.Cg-ACKA.Ec], met14::[HIS3.Sba- |
| | PAND.Tca-PYD4.Lk-YDFG.Ec], met14:: [HIS3.Sba-PAND.Tca-PYD4.Lk], |
| | ura3::[PAND.Tca-MSD.Pa]x5, ura3::[PAND.Tca-YDF1]x11, |
| | pdc6::[MET14.Sba-PDUP.Sen-PDUP.Sen-PDUP.Sen-ACR.Rp-HPCD.Rp- |
| | HPCD.Rp-PCT.Cp-PCT.Cp-PCT.Cp], leu2::[MET14.Sba-RS-IPDH1.Cbe], |
| | pyk1::[LEU2.Kl-PEPCK.Ec-AAT2.Sc-PEPCK.Ec] |

YS_009 recombinant yeast strain was assayed in a 0.7 L bioreactor in the presence of 0.2 L YPD medium fed with about 250 g/L glucose. Stirring was maintained at 500 rpm with a 0.125 vvm aeration just at the very beginning of the fermentation. GC-MS/FID was used to measure ethanol, 1-propanol, acetone, 2-propanol and glucose, and the results are shown in Table 21.

TABLE 21
Co-production of 1-propanol and 2-propanol with ethanol as
a major component during ethanol fermentation from glucose.

| | Added | Consumed | | | | | |
|---|---|---|---|---|---|---|---|
| Time | glucose | glucose | | 1-Propanol | Acetone | 2-Propanol | Ethanol |
| (h) | (g/L) | (g/L) | OD600 nm | (g/L) | (g/L) | (g/L) | (g/L) |
| 07 | 20 | 16 | 28 | ND | ND | ND | 7 |
| 17 | 106 | 90 | 106 | 0.2 | 0.0 | 0.4 | 41 |
| 32 | 214 | 163 | 142 | 0.3 | 0.0 | 0.9 | 79 |
| 40 | 214 | 200 | 150 | 0.3 | 0.0 | 1.0 | 90 |
| 56 | 249 | 218 | 126 | 0.3 | 0.0 | 1.2 | 101 |

YS_009 recombinant yeast was able to consume most of the glucose fed showing a high cell density reaching an OD600 of 150 at 40 hours of fermentation. YS_009 recombinant yeast was able to produce 1.5 g/L of 1-propanol and 2-propanol along with ethanol at high titer of 101 g/L at 56 hours fermentation time. Further, the majority of the carbon source from glucose was transformed into ethanol and a small part of the carbon source converted into 1-propanol and 2-propanol at non-toxic final concentrations. Glycerol was measured with a final titer of 1.4 g/L at 56 hours fermentation time.

Although the results presented herein were demonstrated using the recombinant yeast strain W303, other Saccharomyces cerevisiae yeast strains including industrial yeasts such as PE-2, CAT-1, BG-1 and Ethanol Red yeast strains, which are widely used in industrial sugarcane-ethanol and corn-ethanol fermentation processes, can also be used.

### Example 13: Recombinant Ethanol-Producing Yeast Co-Producing Acrylic Acid with Ethanol as a Major Component During Ethanol Fermentation from Glucose

An ethanol-producing S. cerevisiae yeast strain is genetically modified to co-produce acrylic acid with ethanol as a major component through a carbon flow redirection from glucose as a carbon source. An acrylic acid biosynthetic metabolic pathway via 3-hydroxypropionic acid and target enzymes are heterologously expressed into the laboratory yeast strain W303, and also into the industrial ethanol producer yeast strains, PE-2 and Red strains. Additionally, the yeast strains are modified to downregulate the natural ethanol-producing metabolic pathway in the pyruvate node.

The recombinant yeast strains have the acrylic acid producing pathway genes integrated into the genome, including AAT2 from S. cerevisiae (AAT2.Sc), PAND from T. castaneum (PAND.Tca), PYD4 from L. kluyveri (PYD4.Lk), YDFG from E. coli (YDFG.Ec), HPD1 from C. albicans (HPD1.Ca), PCT from C. propionicum (PCT.Cp), HPCD from R. pomeroyi (HPCD.Rp), and the acyl-CoA hydrolase YciA from E. coli (YciA.Ec). All the acrylic acid biosynthetic pathway genes are codon-optimized to be optimally expressed in yeast, under the control of promoters of varied strengths and also varying the number of gene copies.

These recombinant yeast strains also have PEP.CK from E. coli (PEPCK.Ec) over-expressed to redirect carbon flow from PEP to oxaloacetate (OAA) and optionally have a PYK1 enzyme downregulated using promoters of varied strengths, preferably weak promoters, to decrease PYK1 enzyme half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. Acrylic acid, ethanol, glycerol and glucose are measured after 48 hours fermentation using standard equipment and analytical methods. Acrylic acid is co-produced with ethanol as a major component in a g/L range.

### Example 14: Recombinant Ethanol-Producing Yeast Co-Producing Propionic Acid with Ethanol as a Major Component During Ethanol Fermentation from Glucose

An ethanol-producing S. cerevisiae yeast strain is genetically modified to co-produce propionic acid with ethanol as a major component through a carbon flow redirection from glucose as a carbon source. A propionic acid biosynthetic metabolic pathway via 3-hydroxypropionic acid and target enzymes are heterologously expressed into the W303 yeast strain, and also into the industrial ethanol producer yeast strains PE-2 and Ethanol Red. Additionally, the yeast strains are modified to downregulate the natural ethanol-producing metabolic pathway in the pyruvate node.

The recombinant yeast strain has the propionic acid producing pathway genes integrated into the genome, including AAT2 from S. cerevisiae (AAT2.Sc), PAND from T. castaneum (PAND.Tca), PYD4 from L. kluyveri (PYD4.Lk), YDFG from E. coli (YDFG.Ec), HPD1 from C. albicans (HPD1.Ca), PCT from C. propionicum (PCT.Cp), HPCD from R. pomeroyi (HPCD.Rp), and ACR from R. pomeroyi (ACR.Rp), where PCT.Cp is responsible for CoA-activation of 3-hydroxypropionic acid and the CoA transference from propionyl-CoA to other molecule releasing propionic acid. All the propionic acid biosynthetic pathway genes are codon-optimized to be optimally expressed in yeast, under the control of promoters of varied strengths and also varying the number of gene copies.

These recombinant yeast strains have PEP.CK from E. coli (PEPCK.Ec) over-expressed to redirect carbon flow from PEP to oxaloacetate (OAA) and optionally also have a PYK1 enzyme downregulated using a weak promoter to decrease PYK1 enzyme half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. Propionic acid, ethanol, glycerol and glucose are measured after 48 hours fermentation using standard equipment and analytical methods. 5 g/L, 10 g/L, 15 g/L or more of propionic acid is produced with ethanol as a major competent.

### Example 15: Recombinant Ethanol-Producing Yeast Co-Producing Butanone with Ethanol as a Major Component During Ethanol Fermentation from Glucose

An ethanol-producing S. cerevisiae yeast strain is genetically modified to co-produce butanone with ethanol as a major component through a carbon flow redirection from glucose as a carbon source. Butanone can be produced via propionyl-CoA and acetyl-CoA condensation, wherein both intermediates are derived from malonate semialdehyde. This biosynthetic metabolic pathway and target enzymes are heterologously expressed into the W303 strain, and also into the widely used industrial ethanol producer yeast strains, PE-2 and Red strains. Additionally, the yeast strains are modified to downregulate the natural ethanol-producing metabolic pathway in the pyruvate node.

These recombinant yeast strains have the butanone producing pathway genes integrated into the genome, including AAT2 from S. cerevisiae (AAT2.Sc), PAND from T. castaneum (PAND.Tca), PYD4 from L. kluyveri (PYD4.Lk), YDFG from E. coli (YDFG.Ec), HPD1 from C. albicans (HPD1.Ca), PCT from C. propionicum (PCT.Cp), HPCD and ACR from R. pomeroyi (HPCD.Rp and ACR.Rp), MSD from C. albicans or P. aeruginosa (MSD.Pa or MSD.Ca), the b-ketothiolase BktB from C. necator (BtkB.Cn), ATOAD from E. coli (ATOA.Ec and ATOD.Ec), and ADC from C. acetobutylicum or P. polymyxa (ADC.Ca or ADC.Pp). All the butanone biosynthetic pathway genes are codon-optimized to be optimally expressed in yeast, under the control of promoters of varied strengths and also varying the number of gene copies.

These recombinant yeast strains have PEP.CK from E. coli (PEPCK.Ec) over-expressed to redirect carbon flow from PEP to oxaloacetate (OAA) and optionally also have the PYK1 enzyme downregulated using a weak promoter to decrease its half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. Butanone, ethanol, glycerol and glucose are measured after 48 hours fermentation using standard equipment and analytical methods. 5 g/L, 10 g/L, 15 g/L or more g/L of butanone is co-produced with ethanol as the major component.

### Example 16: Recombinant Ethanol-Producing Yeast Co-Producing 2-Butanol with Ethanol as a Major Component During Ethanol Fermentation from Glucose

An ethanol-producing S. cerevisiae yeast strain is genetically modified to co-produce 2-butanol with ethanol as a major component through a carbon flow redirection from glucose as a carbon source. 2-Butanol can be produced from a MSA-derived butanone as described in the previous example. The 2-butanol biosynthetic metabolic pathway and target enzymes are heterologously expressed into the W303 yeast strain, and also into the widely used industrial ethanol producer yeast strains, PE-2 and Ethanol Red strains. Additionally, the yeast strains are modified to downregulate the natural ethanol-producing metabolic pathway in the pyruvate node.

These recombinant yeast strains have the 2-butanol producing pathway genes integrated into the genome, including AAT2 from S. cerevisiae (AAT2.Sc), PAND from T. castaneum (PAND.Tca), PYD4 from L. kluyveri (PYD4.Lk), YDFG from E. coli (YDFG.Ec), HPD1 from C. albicans (HPD1.Ca), PCT from C. propionicum (PCT.Cp), HPCD and ACR from R. pomeroyi (HPCD.Rp and ACR.Rp), MSD from C. albicans or P. aeruginosa (MSD.Pa or MSD.Ca), the b-ketothiolase BktB from C. necator (BtkB.Cn), ATOAD from E. coli (ATOA.Ec and ATOD.Ec), ADC from C. acetobutylicum or P. polymyxa (ADC.Ca or ADC.Pp), and the secondary alcohol dehydrogenase ADH from L. brevis (ADH.Lb). All the 2-butanol biosynthetic pathway genes are codon-optimized to be optimally expressed in yeast, under the control of promoters of varied strengths and also varying the number of gene copies.

These recombinant yeast strains also have PEP.CK from E. coli (PEPCK.Ec) over-expressed to redirect carbon flow from PEP to oxaloacetate (OAA) and optionally also have a PYK1 enzyme downregulated using a weak promoter to decrease its half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. 2-Butanol, ethanol, glycerol and glucose are measured after 48 hours fermentation using standard equipment and analytical methods. 5 g/L, 10 g/L, 15 g/L or more g/L of 2-butanol is co-produced with ethanol as the major component.

### Example 17: Recombinant Ethanol-Producing Yeast Co-Producing Propyl Acetate with Ethanol as a Major Component During Ethanol Fermentation from Glucose

An ethanol-producing S. cerevisiae yeast strain is genetically modified to co-produce propyl acetate with ethanol as a major component through a carbon flow redirection from glucose as a carbon source. Propyl acetate can be produced by the esterification of 1-propanol and acetyl-CoA. A propyl acetate biosynthetic metabolic pathway and target enzymes are heterologously expressed into the W303 strain, and also into the industrial ethanol producer yeast strains, PE-2 and Ethanol Red strains. Additionally, the yeast strains are modified to downregulate the natural ethanol-producing metabolic pathway in the pyruvate node.

These recombinant yeast strain have the propyl acetate producing pathway genes integrated into the genome, including AAT2 from S. cerevisiae (AAT2.Sc), PAND from T. castaneum (PAND.Tca), PYD4 from L. kluyveri (PYD4.Lk), YDFG from E. coli (YDFG.Ec), HPD1 from C. albicans (HPD1.Ca), PCT from C. propionicum (PCT.Cp), HPCD and ACR from R. pomeroyi (HPCD.Rp and ACR.Rp), MSD from C. albicans or P. aeruginosa (MSD.Pa or MSD.Ca), PDUP from S. enterica (PDUP.Sen), ADH1 from S. cerevisiae (ADH1.Sc), MSD from C. albicans or P. aeruginosa (MSD.Ca or MSD.Pa), and the alcohol β-acetyltransferase 1 ATF1 from S. cerevisiae (ATF1.Sc). All the propyl acetate biosynthetic pathway genes are codon-optimized to be optimally expressed in yeast, under the control of promoters of varied strengths and also varying the number of gene copies.

These recombinant yeast strains have PEP.CK from E. coli (PEPCK.Ec) over-expressed to redirect carbon flow from PEP to oxaloacetate (OAA) and optionally also have a PYK1 enzyme downregulated by using a weak promoter such as pMET25DF or pNUP57 to decrease its half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. Propyl acetate, ethanol, glycerol and glucose are measured after 48 hours fermentation using standard equipment and analytical methods. Propyl acetate is co-produced with ethanol as the major component in a g/L range.

### Example 18: Recombinant Ethanol-Producing Yeast Co-Producing 2,3-Butanediol with Ethanol as a Major Component During Ethanol Fermentation from Glucose

An ethanol-producing S. cerevisiae yeast strain is genetically modified to co-produce 2,3-butanediol with ethanol as a major component through a carbon flow redirection from glucose as a carbon source. A 2,3-butanediol biosynthetic metabolic pathway and target enzymes are heterologously expressed into the W303 strain, and also into the industrial ethanol producer yeast strains, PE-2 and Red strains.

These recombinant yeast strains have the 2,3-butanediol producing pathway genes integrated into the genome, including the acetolactate synthase ALS from P. polymyxa (ALS.Pp), the acetolactate decarboxylase from B. subtilis (ALD.Bs) and the 2,3-butanediol dehydrogenase from C. autoethanogenum (BDH.Ca). All the 2,3-butanediol biosynthetic pathway genes are codon-optimized to be optimally expressed in yeast under the control of promoters of varied strengths and also varying the number of gene copies. Beyond the expression of enzymes that compete for the same substrate, pyruvate, the carbon flow can be even more diverted from ethanol to 2,3-butanediol by a genetic manipulation that reduces the activity of pyruvate decarboxylase (PDC) like the use of weaker promoters and/or the deletion of one or more isoenzymes.

A fermentation test is performed in the presence of 25 mL of YPD media with 80 g/L glucose in 125 mL fermentation flask. Stirring is maintained at 135 rpm on 50 mm shaking diameter incubators at 30-35° C. 2,3-Butanediol, ethanol, glycerol and glucose are measured after 48 hours fermentation using standard equipment and analytical methods. 5 g/L, 10 g/L, 15 g/L or more g/L of 2,3-Butanediol is co-produced with ethanol as the major component.

### Example 19: Recombinant Ethanol-Producing Yeast Co-Producing Succinic Acid with Ethanol as a Major Component During Ethanol Fermentation from Glucose

An ethanol-producing S. cerevisiae yeast strain is genetically modified to co-produce succinic acid with ethanol as a major component through a carbon flow redirection from glucose as a carbon source. A succinic acid biosynthetic metabolic pathway and target enzymes are heterologously expressed into the laboratory yeast strain W303, and also into the industrial ethanol producer yeast strains PE-2 and Red strains. Additionally, the yeast strains are modified to downregulate the natural ethanol-producing metabolic pathway in the pyruvate node.

These recombinant yeast strains have the succinic acid producing pathway genes integrated into the genome including the malate dehydrogenase Mdh from R. delemar (MDH.Rd), the fumarase FumC and the fumarate reductase FumABCD from E. coli (FUMC.Ec and FUMABCD.Ec). All the heterologous genes are codon-optimized to be optimally expressed in yeast under the control of promoters of varied strengths and also varying the number of gene copies.

These recombinant yeast strains also have PEP.CK from E. coli (PEPCK.Ec) over-expressed to redirect carbon flow from PEP to oxaloacetate (OAA) and optionally also have a PYK1 enzyme downregulated by using a weak promoter such as pMET25DF to decrease its half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. Succinic acid, ethanol, glycerol and glucose are measured after 48 hours fermentation using standard equipment and analytical methods. Succinic acid is co-produced with ethanol as a major component in a g/L range.

### Example 20: Recombinant Ethanol-Producing Yeast Co-Producing 1,4-Butanediol with Ethanol as a Major Component During Ethanol Fermentation from Glucose

An ethanol-producing S. cerevisiae yeast strain is genetically modified to co-produce 1,4-butanediol with ethanol as a major component through a carbon flow redirection from glucose as a carbon source. A 1,4-Butanediol biosynthetic metabolic pathway and target enzymes are heterologously expressed into the W303 strain, and also into the industrial ethanol producer yeast strains like PE-2, BG-1, CAT-1 and Red strains, with a subsequent downregulation of the natural ethanol-producing metabolic pathway in the pyruvate node as demonstrated.

These recombinant yeast strains have the 1,4-butanediol producing pathway genes integrated into the genome, including the malate dehydrogenase Mdh from R. delemar (MDH.Rd), the fumarase FumC, the fumarate reductase FumABCD and the succinyl-CoA synthetase SucCD from E. coli (FUMC.Ec, FUMABCD.Ec and SUCCD.Ec), the CoA-dependent succinate semialdehyde dehydrogenase SucD, the 4-hydroxybutyrate dehydrogenase 4bdh and the CoA-acyl transferase Cat2 from P. gingivalis (SUCD.Pg, 4HBDH.Pg and CAT2.Pg), the CoA-dependent aldehyde dehydrogenase ALD and alcohol dehydrogenase ADH from C. acetobutylicum (ALD.Ca and ADH.Ca). All the 1,4-butanediol biosynthetic pathway genes are codon-optimized to be optimally expressed in yeast under the control of promoters of varied strengths and also varying the number of gene copies.

These recombinant yeast strains have PEP.CK from E. coli (PEPCK.Ec) over-expressed to redirect carbon flow from PEP to oxaloacetate (OAA) and optionally also have a PYK1 enzyme downregulated by using a weak promoter such as pMET25DF to decrease its half-life and thereby reduce the carbon flow from PEP towards pyruvate and better control the amount of ethanol naturally produced.

A fermentation test is performed in the presence of 25 mL of YPD media with 80 g/L glucose in 125 mL fermentation flask. Stirring is maintained at 135 rpm on 50 mm shaking diameter incubators at 30-35° C. 1,4-butanediol, ethanol, glycerol and glucose are measured after 48 hours fermentation using standard equipment and analytical methods. 1,4-butanediol is co-produced with ethanol in a g/L range.

### Example 21: Recombinant Ethanol-Producing Yeast Co-Producing One or More Co-Products During Industrial Ethanol Fermentation Conditions Based on Industrial Sugarcane Raw Material

An industrial ethanol-producing S. cerevisiae yeast strain is genetically modified to produce ethanol with one or more co-products during industrial ethanol fermentation processes from sugarcane raw material as a carbon source. This is preferably an industrial ethanol-producing S. cerevisiae strain already used industrially on sugarcane-ethanol fermentation processes including PE-2, BG-1, CAT-1 strains.

This genetically modified S. cerevisiae yeast strain is obtained as described in previous examples to be capable of producing ethanol with one or more co-products at non-toxic concentrations. This genetically modified S. cerevisiae yeast strain is capable of producing ethanol with 1-propanol, acetone, 2-propanol or a combination thereof. This genetically modified S. cerevisiae yeast strain is capable of producing ethanol with 1-propanol, acetone, 2-propanol or a combination thereof at non-toxic concentrations for the industrial ethanol-producing yeast strain, PE-2, BG-1 and CAT-1 strain.

This genetically modified S. cerevisiae yeast strain is capable of co-producing ethanol with 1-propanol, acetone, 2-propanol or a combination thereof from an industrial sugarcane material through small-scale fermentation tests that mimic an industrial sugarcane-ethanol fermentation condition. This genetically modified S. cerevisiae yeast strain is tested on a 500 mL using 200 mL of cane molasses solution 170 g/L of TRS (total reduced sugars). 140 mL of molasses solution is mixed with 70 mL of yeast suspension (the inoculum) containing around 100 g/L (DWC). The flask is plugged with an airlock type S (to promote anaerobic conditions). Then, the culture is carried out at 32° C., 150 rpm and during 8 h. At the end of the culture, the beer is centrifugate and yeast pellet is separated from the clarified beer. The yeast pellet is resuspended with 74 mL of the clarified beer. Samples are taken from the clarified beer and from the resuspended yeast. Then, a new cycle is started by mixing 140 mL of molasses solution (170 g/L TRS) with the 70 mL of resuspended yeast (4 ml was used as samples). This procedure is repeated during 20 cycles. Samples at end of each fermentation are taken for HPLC, GC-MS/FID and standard analytical methods know by someone skilled in the Art. Glucose, sucrose, ethanol, glycerol, 1-propanol, acetone and 2-propanol are measured. This genetically modified S. cerevisiae yeast strain shows quite similar industrial ethanol fermentation robustness and performance (such as ethanol yield and titer) expected for its mother industrial ethanol-producing yeast strain, PE-2, BG-1 and CAT-1. The alcohols yield is around 0.43 to 0.46 grams of total alcohols produced per gram of sugar, meanwhile total ethanol titer is around 60-80 g/L. Ethanol is present in an amount of around 80-85% wt. based on a total weight of produced alcohols. On the other hand, a total concentration of alcohols (n-propanol, 2-propanol and acetone) attained is around 15%-20% wt. This result demonstrates the process of producing industrial ethanol-producing yeast, genetically modified to enable its use for the production of ethanol as a major component with 1-propanol, acetone and/or 2-propanol at non-toxic concentrations, without compromising its mother yeast robustness and fermentation performance adequate for industrial production applications.

### Example 22: Recombinant Ethanol-Producing Yeast Co-Producing One or More Co-Products During Industrial Ethanol Fermentation Conditions Based on Industrial Corn Raw Material

An industrial ethanol-producing S. cerevisiae yeast strain is genetically modified to produce ethanol with one or more co-products during industrial ethanol fermentation processes from corn raw material as a carbon source. This is preferably an industrial ethanol-producing S. cerevisiae strain already used industrially on corn-ethanol fermentation processes like Ethanol Red® (Leaf-Lesaffre) strain.

This genetically modified S. cerevisiae yeast strain is obtained as described in previous examples to be capable of producing ethanol with one or more co-products at non-toxic concentrations. This genetically modified S. cerevisiae yeast strain is capable of producing ethanol with 1-propanol, acetone, 2-propanol or a combination thereof. This genetically modified S. cerevisiae yeast strain is capable of producing ethanol with 1-propanol, acetone, 2-propanol or a combination thereof at non-toxic concentrations for the industrial ethanol-producing yeast strain, such as Ethanol Red® (Leaf-Lesaffre) strain.

This genetically modified S. cerevisiae yeast strain is capable of co-producing ethanol with 1-propanol, acetone, 2-propanol or a combination thereof from an industrial corn material through small-scale fermentation tests that mimic an industrial corn-ethanol fermentation condition. This genetically modified S. cerevisiae yeast strain is tested in a 3.5 L bioreactor using 1 L of partially hydrolyzed corn mash. An adequate dose of glucoamylase enzyme is added and 0.5 g/L fresh yeast is inoculated. Initial pH is adjusted to 4.5 but there is no control during the fermentation. Temperature is set at 35° C. with 300 rpm stirring. The culture is carried out during 72 h and samples are taken at proper intervals. The experiment is performed in triplicate.

HPLC, GC-MS/FID and other standard analytical methods are used to measure sugars, glucose, ethanol, glycerol, 1-propanol, acetone and 2-propanol. This genetically modified S. cerevisiae yeast strain shows quite similar industrial ethanol fermentation robustness and performance (such as ethanol yield and titer) compared to the industrial ethanol-producing yeast strains. The alcohols yield is around 0.43 to 0.46 grams of total alcohols produced per gram of sugar; meanwhile, the total alcohols titer is around 120-150 g/L. Ethanol is present in an amount of around 80-85% wt. based on a total weight of produced alcohols. On the other hand, a total concentration of alcohols (n-propanol, 2-propanol and acetone) is attained around 15%-20% wt. This result demonstrates the process of producing industrial ethanol-producing yeast, genetically modified to enable its use for the production of ethanol as a major component with 1-propanol, acetone and/or 2-propanol at non-toxic concentrations, without compromising its mother yeast robustness and fermentation performance adequate for industrial production applications.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein can be further limited in the claims using consisting of and/or consisting essentially of language. Embodiments of the disclosure so claimed are inherently or expressly described and enabled herein.

It is to be understood that the embodiments of the disclosure disclosed herein are illustrative of the principles of the present disclosure. Other modifications that can be employed are within the scope of the disclosure. Thus, by way of example, but not of limitation, alternative configurations of the present disclosure can be utilized in accordance with the teachings herein. Accordingly, the present disclosure is not limited to that precisely as shown and described.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A process for the production of ethanol and one or more co-products comprising:
(a) contacting a fermentable carbon source with an ethanol-producing yeast in a fermentation medium;
(b) fermenting the yeast in the fermentation medium, wherein the yeast produces ethanol and one or more co-products from the fermentable carbon source, wherein the produced ethanol is present in a greater concentration in mg/mL than the produced co-products; and
(c) isolating the ethanol and the one or more co-products;
wherein the yeast is a recombinant yeast genetically modified to produce the one or more co-products.

2. The process of claim 1, wherein the carbon source is glucose or dextrose.

3. The process of claim 1, wherein the carbon source is derived from renewable grain sources obtained by saccharification of a starch-based feedstock, such as corn, wheat, rye, barley, oats, rice, or mixtures thereof.

4. The process of claim 1, wherein the carbon source is from a renewable sugar, such as sugar cane, sugar beets, cassava, sweet sorghum, or mixtures thereof.

5. The process of claim 1, wherein the ethanol-producing yeast is Saccharomyces cerevisiae.

6. The process of claim 5, wherein the Saccharomyces cerevisiae is an industrial strain, any common strain used in ethanol industry, a typical laboratory strain, or any strain resulting from the typical method of crossing between strains.

7. The process of claim 1, wherein the co-products are produced at non-toxic concentrations for the ethanol-producing yeast.

8. The process of claim 1, wherein the produced ethanol is present in an amount of at least 70 wt. % based on a total weight of produced ethanol and co-products, such as at least 75 wt. %, at least 80 wt. %, at least 85 wt. %, at least 90 wt. %, or at least 95 wt. %.

9. The process of claim 1, wherein the fermentation is carried out as a batch process, a fed batch process, or a continuous process.

10. The process of claim 1, wherein the fermentation is carried out under anaerobic conditions for about 24 to about 96 hours at a temperature of about 15° C. to about 60° C.

11. The process of claim 1, wherein the fermentation is carried out in an industrial ethanol plant, preferably in an already-existing industrial ethanol plant.

12. The process of claim 1, wherein the one or more co-products are selected from the group consisting of an alcohol other than ethanol; a ketone; a glycol; an ether; an ester; a diamine; a carboxylic acid; an amino acid; a diene, and an alkene.

13. The process of claim 1, wherein the one or more co-products are selected from the group consisting of 1-butanol, 2-butanol, isobutanol, methanol, n-propanol, isopropanol, isoamyl alcohol, acetone, methyl ethyl ketone, methyl propionate, 1,3-propanediol, monoethylene glycol, propylene glycol, citric acid, lactic acid, succinic acid, adipic acid, acetic acid, glutamic acid, propionic acid, furan dicarboxylic acid, 2,4 furandicarboxylic acid, 2,5-furandicarboxylic acid, 3-hydroxypropionic acid, acrylic acid, itaconic acid, glutamic acid, ethyl acetate, isopropyl acetate, propyl acetate, isoprenol, 1,3-butanediol, 1,4-butanediol, 2,3-butanediol, diethanolamine, tryptophan, threonine, methionine, lysine, serine, tyrosine, butadiene, isoprene, ethane, and propene.

14. The process of claim 1, wherein isolating the ethanol and the one or more co-products comprises a process selected from distillation, adsorption, crystallization, absorption, electrodialysis, solvent extraction, ion exchange resin chromatography, evaporation, or a combination thereof.

15. A process for the production of ethanol and one or more co-products comprising:
(a) contacting a fermentable carbon source with an ethanol-producing yeast in a fermentation medium;
(b) fermenting the yeast in the fermentation medium, wherein the yeast produces ethanol and one or more low boiling co-products from the fermentable carbon source, wherein the produced ethanol is present in a greater concentration in mg/mL than the produced co-products; and
(c) isolating the ethanol and the one or more low boiling co-products;
wherein the yeast is a recombinant yeast genetically modified to produce the one or more co-products.

16. The process of claim 15, wherein the low boiling co-products have, at a standard pressure of 100 kPa (1 bar), a boiling point of 100° C. or less, such as 99° C. or less, 98° C. or less, 97° C. or less, 95° C. or less, 90° C. or less, 85° C. or less, 80° C. or less, 75° C. or less, 70° C. or less, 65° C. or less, or 60° C. or less.

17. The process of claim 15, wherein the one or more low boiling co-products are selected from acetone, 1-propanol, 2-propanol, or a combination thereof.

18. The process of claim 15, wherein isolating the ethanol and the one or more low boiling co-products is conducted by sequential distillation units.

19. A process for the production of ethanol and one or more co-products comprising:
(a) contacting a fermentable carbon source with an ethanol-producing yeast in a fermentation medium;
(b) fermenting the yeast in the fermentation medium, wherein the yeast produces ethanol and one or more high boiling co-products from the fermentable carbon source, wherein the produced ethanol is present in a greater concentration in mg/mL than the produced co-products; and
(c) isolating the ethanol and the one or more high boiling co-products;
wherein the yeast is a recombinant yeast genetically modified to produce the one or more high boiling co-products.

20. The process of claim 19, wherein the high boiling co-products have, at a standard pressure of 100 kPa (1 bar), a boiling point of more than 100° C., such as more than 105° C., more than 110° C., more than 120° C., more than 130° C., more than 140° C., more than 150° C., more than 160° C., more than 170° C., more than 180° C., more than 190° C., more than 200° C., more than 210° C., more than 220° C., more than 230° C., more than 240° C., or more than 250° C.

21. The process of claim 19, wherein isolating the ethanol and the one or more high boiling co-products is conducted by distillation and followed by a process selected from crystallization, solvent extraction, chromatographic separation, salt splitting, sedimentation, acidification, ion exchange, evaporation, or combinations thereof.
